(19) United States
(12) Patent Application Publication
(10) Pub. No.: US 2015/0232407 A1
(43) Pub. Date: Aug. 20, 2015

(54) DRUG SUBSTANCE PREPARATIONS, PHARMACEUTICAL COMPOSITIONS AND DOSAGE FORMS COMPRISING S-(+)-FLURBIPROFEN

(71) Applicant: Aesica Pharmaceuticals Limited, Newcastle upon Tyne (GB)

(72) Inventors: Alan Michael Raymond, Rowley (GB); Barrie Rhodes, Newcastle upon Tyne (GB)

(21) Appl. No.: 14/429,607

(22) PCT Filed: Sep. 20, 2013
(86) PCT No.: PCT/GB2013/052464

§ 371 Date: Mar. 19, 2015

(30) Foreign Application Priority Data
Sep. 21, 2012 (GB) 1216893.6

(51) Int. Cl. C07C 57/38

(52) U.S. Cl. CPC C07C 57/38; C07B 2200/07

(57) ABSTRACT

There is described (S)-(+)-2-(2-fluoro-4-biphenylyl) propionic acid, or a salt or ester thereof, having substantially limited amounts of specific impurities associated with the synthesis and purification of the (S)-(+)-2-(2-fluoro-4-biphenylyl) propionic acid.

### FIELD OF THE INVENTION

The present invention relates to pharmaceutical compositions, drug substance preparations, pharmaceutical compositions, and dosage forms.

### BACKGROUND OF THE INVENTION

The development of all pharmaceutical products requires the production of high purity drug substance preparations, pharmaceutical compositions and dosage forms that contain effective amounts of the active pharmaceutical ingredient, and minimal impurities. However, pharmaceutical products containing an active ingredient that must be dosed in large amounts for long periods of time, present a special challenge to drug manufacturers since, in such cases, the potential exposure of the patient to any impurities present in the pharmaceutical product is amplified.

All drug substance preparations, regardless of the active ingredient, contain finite amounts of impurities. These impurities can generally be grouped into categories based upon their chemical identity.

Impurities that are structurally similar to the active ingredient are commonly referred to as “product-related impurities”. In the case of active ingredients containing chiral centres where one enantiomer shows therapeutic effect, while the other enantiomer shows either no effect, minimal effect, or an undesirable effect, the latter enantiomer represents a type of product-related impurity, commonly referred to as an “enantiomeric impurity.”

Impurities that are not structurally similar to the active ingredient, and are introduced by the process(es) used to make the active ingredient, are commonly referred to as “process-related impurities.” Process-related impurities can comprise such things as unreacted starting materials, materials added to purify the active ingredient, by-products of side reactions, and the like, which do not structurally resemble the active ingredient. Process-related impurities may also comprise residual solvents and heavy metals. However, due to their known toxic properties, residual solvents and heavy metals are often considered apart from other types of process-related impurities.

Since drug substance preparations containing a given active ingredient are used to prepare pharmaceutical compositions, which, in turn are used to manufacture drug products for administration of the active ingredient, pharmaceutical compositions and drug products generally contain the product- and process-related impurities that co-occur with the given active ingredient in the starting drug substance preparations. In order to minimize risk to human health caused by impurities within drug products, governmental agencies establish specific limits for various types of impurities that arise in final drug products from the active ingredient-containing drug substance preparations. Drug substance preparations and excipients must typically have impurity levels that are equal to or less than these limits, in order for manufacturers to gain governmental approval to market and sell their drug products. 2-(2-fluoro-4-biphenylyl) propionic acid (flurbiprofen) is generally commercially available as the racemate, i.e. (R,S)-(±)-2-(2-fluoro-4-biphenylyl) propionic acid (R,S)-(±)-flurbiprofen), i.e. a mixture of enantiomers (R)-(−)-flurbiprofen and (S)-(+)-flurbiprofen. (R,S)-(±)-flurbiprofen is commercially available as a non-steroidal anti-inflammatory drug (NSAID) which is therapeutically useful in the treatment or alleviation of, inter alia, inflammation and pain. However, in the racemate, (R,S)-(±)-flurbiprofen, it is the (S)-(+)-enantiomer, (S)-(+)-flurbiprofen, that is the therapeutically active species as an NSAID. The (R)-(−)-enantiomer is generally inactive as an NSAID.

As with drug substance preparations of any synthetic organic active ingredient, drug substance preparations of (S)-(+)-2-(2-fluoro-4-biphenylyl) propionic acid (S-(+)-flurbiprofen) can contain product-related impurities (e.g., (R)-(−)-2-(2-fluoro-4-biphenylyl) propionic acid), process-related impurities, residual solvents, and heavy metals.

Since there is an increasing need for pharmaceutical dosage forms containing (S)-(+)-2-(2-fluoro-4-biphenylyl) propionic acid as the active ingredient, and since the active ingredient in these dosage forms may, in some circumstances require administration via a non-enteral route, there is a clear need for pharmaceutical compositions containing (S)-(+)-2-(2-fluoro-4-biphenylyl) propionic acid and dosage forms made from (S)-(+)-2-(2-fluoro-4-biphenylyl) propionic acid containing acceptable levels of impurities of all types. This includes the need to remove or minimise the presence of R)-(−)-2-(2-fluoro-4-biphenylyl) propionic acid) ((R)-(−)-flurbiprofen) since, as an NSAID, it is an inactive species.

Methods of resolving (S)-(+)-2-(2-fluoro-4-biphenylyl) propionic acid from racemic flurbiprofen are disclosed in U.S. Pat. No. 5,599,969 of Hardy et al. (assigned to The Boots Company PLC (Nottingham, GB)). These methods, which have been adapted to the present invention, and are described in detail below, involve reacting racemic flurbiprofen with α-methylbenzylamine to form an isolatable salt of (S)-(+)-2-(2-fluoro-4-biphenylyl) propionic acid.

U.S. Pat. No. 4,209,638 of Nicholson and Tantum (also assigned to The Boots Company PLC (Nottingham, GB)) discloses a process for resolving 2-arylpropionic acids, which include flurbiprofen, by mixing the racemate with a chiral organic nitrogenous base under certain conditions followed by recovery and separation of the diastereomeric salts.

Other patents disclosing processes for resolving racemic arylpropionic acids include U.S. Pat. No. 4,983,765 (assigned to PAZ Arzneimittel-Entwicklungsgesellschaft mbH (Frankfurt am Main, DE)); U.S. Pat. No. 5,015,764 (assigned to Ethyl Corporation (Richmond, Va.)); U.S. Pat. No. 5,235,100 (also assigned to Ethyl Corporation (Richmond, Va.)); U.S. Pat. No. 5,574,183 (assigned to Albemarle Corporation (Richmond, Va.); and U.S. Pat. No. 5,510,519 (assigned to Sumitomo Chemical Company, Limited (Osaka, JP)).

International patent application No. PCT/US2008/052853—Myriad, discloses drug substance preparations containing (R)-(−)-2-(2-fluoro-4-biphenylyl) propionic acid.

Importantly, the inventors have discovered drug substance preparations having (S)-(+)-2-(2-fluoro-4-biphenylyl) propionic acid, or a salt or ester thereof, as the active ingredient, and containing substantially limited amounts of specific product-related and process-related impurities, residual solvents and heavy metals. The inventive pharmaceutical compositions and drug substance preparations allow for the production of pharmaceutical compositions that further allow for the production of dosage forms having (S)-(+)-2-(2-fluoro-4-biphenylyl) propionic acid, or a salt or ester thereof, as the active ingredient, and substantially limited quantities of specific impurities. These pharmaceutical compositions and dosage forms are particularly well suited for use in treating (and/or preventing) conditions or diseases, like, for instance, inter alia, inflammation and pain, for example, rheumatoid arthritis, osteoarthritis, postoperative pain and soft tissue injuries.

It is important to use drug substance preparations of high purity, and with substantially limited amounts of specific product- and process-related impurities, residual solvents, and heavy metals, in the preparation of pharmaceutical compositions and dosage forms in order to minimize the exposure of patients to these impurities, and thereby minimize the likelihood of any deleterious effects that may be caused by such impurities. Consequently, the inventors have also discovered pharmaceutical compositions containing (S)-(+)-2-(2-fluoro-4-biphenylyl) propionic acid, or a salt or ester thereof, that allow for the production of oral dosage forms such as, for example, tablets having 25 mg or more of (S)-(+)-2-(2-fluoro-4-biphenylyl) propionic acid, or a salt or ester thereof, and substantially limited amounts of specific impurities. These pharmaceutical compositions, and the dosage forms comprising them, have desirable physical, mechanical and manufacturing properties, requisite stability, excellent dissolution profiles, and therapeutically desirable pharmacokinetic profiles.

### SUMMARY OF THE INVENTION

The invention relates to S)-(+)-2-(2-fluoro-4-biphenylyl) propionic acid, or a salt or ester thereof, and to drug substance preparations having (S)-(+)-2-(2-fluoro-4-biphenylyl) propionic acid, or a salt or ester thereof, as the active ingredient, and having substantially limited amounts of specific impurities associated with the synthesis and purification of the active ingredient.

The invention further relates to pharmaceutical compositions made from (S)-(+)-2-(2-fluoro-4-biphenylyl) propionic acid, or a salt or ester thereof, and dosage forms or drug products made from these pharmaceutical compositions.

The pharmaceutical compositions and the dosage forms of the invention are formulated with (S)-(+)-2-(2-fluoro-4-biphenylyl) propionic acid, or a salt or ester thereof, and one or more pharmaceutically acceptable excipients (inactive pharmaceutical ingredients), in admixture with the (S)-(+)-2-(2-fluoro-4-biphenylyl) propionic acid, or a salt or ester thereof. The pharmaceutical compositions and dosage forms of the invention are specifically formulated for administration of the active ingredient to patients in need of such treatment, such as embodiments formulated for oral administration (e.g. a tablet dosage form).

The invention relates to drug substance preparations, pharmaceutical compositions, and dosage forms that contain (S)-(+)-2-(2-fluoro-4-biphenylyl) propionic acid (S-(+)-flurbiprofen), or a salt or ester thereof, as the active ingredient, and contain limited amounts of specific impurities. The inventors have discovered drug substance preparations of (S)-(+)-2-(2-fluoro-4-biphenylyl) propionic acid having minimal impurities that allow for the production of pharmaceutical dosage forms or drug products containing (S)-(+)-2-(2-fluoro-4-biphenylyl) propionic acid that have desirable physical characteristics, that produce therapeutically desirable pharmacokinetic profiles in human subjects, and that contain limited quantities of product-related impurities, process-related impurities, residual solvents, and heavy metals.

The drug substance preparations, pharmaceutical compositions and dosage forms of the invention are useful for treating a (and/or preventing) a variety of conditions and especially conditions that are treated by the parenteral (non-enteral) administration of (S)-(+)-2-(2-fluoro-4-biphenylyl) propionic acid ((S)-(+)-flurbiprofen), or a salt or ester thereof.

It is important to limit all types of impurities in the dosage forms or drug products given to patients. In particular, it is important to limit those product-related and process-related impurities that are introduced into the dosage form by way of the drug substance preparations that contain the active ingredient and are used to make the pharmaceutical compositions used to manufacture the dosage form or drug product.

Thus, in a first aspect the invention provides a highly pure form of (S)-(+)-2-(2-fluoro-4-biphenylyl) propionic acid ((S)-(+)-flurbiprofen), or a salt or ester thereof. The term “highly pure” will be understood to mean a form of (S)-(+)-2-(2-fluoro-4-biphenylyl) propionic acid ((S)-(+)-flurbiprofen), or a salt or ester thereof, which is chemically pure. This is to be distinguished from entantiomeric purity. Therefore, according to this aspect of the invention there is provided (S)-(+)-2-(2-fluoro-4-biphenylyl) propionic acid, or a salt or ester thereof, having substantially limited amounts of specific impurities associated with the synthesis and purification of the (S)-(+)-2-(2-fluoro-4-biphenylyl) propionic acid.

Thus, highly pure (S)-(+)-2-(2-fluoro-4-biphenylyl) propionic acid ((S)-(+)-flurbiprofen), or a salt or ester thereof, comprises only limited amounts of specific product-related impurities, process-related impurities, residual solvents and/or heavy metals.

According to a further aspect of the invention there is provided a pharmaceutical composition comprising (S)-(+)-2-(2-fluoro-4-biphenylyl) propionic acid, or a salt or ester thereof, as the active ingredient, and having substantially limited amounts of specific impurities associated with the synthesis and purification of the active ingredient.

In one embodiment of this aspect of the invention, all of the impurities present in S)-(+)-2-(2-fluoro-4-biphenylyl) propionic acid, or a salt or ester thereof, are limited to about ≦5%, ≦4%, ≦3%, ≦2%, ≦1%, ≦0.9%, ≦0.8%, ≦0.7%, ≦0.6%, ≦0.5%, ≦0.4%, ≦0.3%, ≦0.2%, ≦0.1, ≦0.01% or ≦0.001%, based on the total weight of the (S)-(+)-2-(2-fluoro-4-biphenylyl) propionic acid, or a salt or ester thereof. The level of all of the impurities present as hereinbefore described will be understood to be:

[the sum of weight(s) of one or more impurities]×100% [total weight of (S-(+)-flurbiprofen)]

In another embodiment, the invention provides (S)-(+)-2-(2-fluoro-4-biphenylyl) propionic acid, or a salt or ester thereof, containing about ≦2%, ≦1%, ≦0.5%, ≦0.25%, ≦0.1%, ≦0.05%, ≦0.025%, ≦0.01%, ≦0.005% ≦0.0025%, or ≦0.001% of (R)-(+)-2-(2-fluoro-4-biphenylyl) propionic acid, or a salt thereof, as the active ingredient, based on the total weight of the (S-(+)-flurbiprofen), or a salt or ester thereof

In another embodiment, the invention provides (S)-(+)-2-(2-fluoro-4-biphenylyl) propionic acid, or a salt or ester thereof, having from 0.001%-0.01%, 0.005%-0.05%, 0.01%-0.1%, 0.05%-0.5%, or 0.1%-1%, of any one specific impurity, by weight.

In another embodiment, the invention provides (S)-(+)-2-(2-fluoro-4-biphenylyl) propionic acid, or a salt or ester thereof, having from 0.001%-0.01%, 0.005%-0.05%, 0.01%-0.1%, 0.05%-0.5%, 0.1%-1%, or 0.5%-5%, by weight, of the specific product-related impurities, process-related impurities, residual solvents and heavy metals define herein.

In certain embodiments, the present invention comprises drug substance preparations containing (S)-(+)-2-(2-fluoro-4-biphenylyl) propionic acid, or a salt or ester thereof, as hereinbefore defined as the active ingredient, and limited amounts of product-related impurities.

In these embodiments, the product-related impurities that are present in limited amounts include, e.g. the enantiomeric impurity (R)-(−)-2-(2-fluoro-4-biphenylyl) propionic acid (which is also known as (R)-(−)-flurbiprofen), 2-(4-biphenylyl) propionic acid, and methyl(2-(2-fluoro-4-biphenylyl)) propionate.

In specific embodiments, the present invention comprises (S)-(+)-2-(2-fluoro-4-biphenylyl) propionic acid, or a salt or ester thereof, wherein the amounts of 2-(4-biphenylyl) propionic acid, as well as the enantiomeric impurity (R)-(−)-2-(2-fluoro-4-biphenylyl) propionic acid, are limited below certain maximum levels.

In another specific embodiment, the present invention comprises (S)-(+)-2-(2-fluoro-4-biphenylyl) propionic acid, or a salt or ester thereof, wherein the amounts of both 2-(4-biphenylyl) propionic acid and methyl(2-(2-fluoro-4-biphenylyl))propionate, as well as the enantiomeric impurity (R)-(−)-2-(2-fluoro-4-biphenylyl) propionic acid, are limited below certain maximum levels.

In certain embodiments, the present invention comprises (S)-(+)-2-(2-fluoro-4-biphenylyl) propionic acid, or a salt or ester thereof, wherein, in addition to the amounts of one or two known product-related impurities, as well as the enantiomeric impurity (R)-(−)-2-(2-fluoro-4-biphenylyl) propionic acid, being limited below certain maximum levels, the amounts of specific process-related impurities are also limited below certain maximum levels. In these embodiments, the principle process-related impurity that is limited below certain maximum levels is (S)-(−)-α-methylbenzylamine (which is also known as (S)-(−)-1-phenylethylamine), which is used as a chiral crystallization agent to resolve and isolate (S)-(+)-2-(2-fluoro-4-biphenylyl) propionic acid from racemic flurbiprofen (i.e. (R,S)-2-(2-fluoro-4-biphenylyl) propionic acid) or from non-racemic mixtures of (R)-(−)-flurbiprofen and (S)-(+)-flurbiprofen.

In other embodiments, additional process-related impurities that are limited below certain maximum levels include residual solvents, and/or heavy metals. In these embodiments, the residual solvents to be limited include, e.g. toluene, methanol, and n-heptane.

In another embodiment of this aspect of the invention, the present invention comprises (S)-(+)-2-(2-fluoro-4-biphenylyl) propionic acid, or a salt or ester thereof, with limited amounts of three product-related impurities, including the enantiomeric impurity (R)-(−)-2-(2-fluoro-4-biphenylyl) propionic acid, 2-(4-biphenylyl) propionic acid, and methyl(2-(2-fluoro-4-biphenylyl))propionate, limited amounts of the process-related impurity (S)-(−)-α-methylbenzylamine, and limited amounts of residual solvents and heavy metals.

In another aspect, the invention provides a method of resolving racemic flurbiprofen (i.e., (R,S)-2-(2-fluoro-4-biphenylyl) propionic acid) or non-racemic mixtures of (R)-(−)-flurbiprofen and (S)-(+)-flurbiprofen using chiral crystallization, in order to purify the (S)-(+)-2-(2-fluoro-4-biphenylyl) propionic acid, or a salt or ester thereof, and optionally preparing drug substance preparations containing the limited amounts of product-related impurities, process-related impurities, residual solvents and heavy metals, as hereinbefore described.

In another aspect, the invention provides a pharmaceutical composition comprising (S)-(+)-2-(2-fluoro-4-biphenylyl) propionic acid, or a salt or ester thereof in admixture with one or more pharmaceutically acceptable excipients.

Consequently, in an embodiment of this aspect of the invention, the pharmaceutical composition of the invention comprises (S)-(+)-2-(2-fluoro-4-biphenylyl) propionic acid, or a salt or ester thereof, as the active ingredient, and further comprises limited quantities of product-related impurities, process-related impurities, residual solvents and heavy metals as hereinbefore described.

In this embodiment, the present invention provides a pharmaceutical composition in which all of the impurities deriving from the (S)-(+)-flurbiprofen), or a salt or ester thereof, represent about ≦5%, ≦4%, ≦3%, ≦2%, ≦1%, ≦0.5%, ≦0.25% ≦0.1%, ≦0.05%, ≦0.025% or ≦0.01% of the total weight of the pharmaceutical composition, i.e.

[sum of weight(s) of impurities deriving from the (S)-(+)-flurbiprofen]×100% [total weight of pharmaceutical composition]

In certain embodiments of this aspect, the invention provides a pharmaceutical composition containing (S)-(+)-2-(2-fluoro-4-biphenylyl) propionic acid, or a salt or ester thereof, and about ≦2%, ≦1%, ≦0.5%, ≦0.25%, ≦0.1%, ≦0.05%, ≦0.025%, ≦0.01% or ≦0.005% of the total weight of the pharmaceutical composition as impurities arising from the (S)-(+)-flurbiprofen used to prepare the composition.

In another embodiment, the invention provides a pharmaceutical composition having from 0.001%-0.01%, 0.01%-0.1%, or 0.1%-1% of one or more impurities, by weight, wherein the impurities derive from (S)-(+)-flurbiprofen used to prepare the pharmaceutical composition.

In another embodiment, the invention provides a pharmaceutical composition having from 0.001%-0.01%, 0.005%-0.05%, 0.01%-0.1%, 0.05%-0.5%, 0.1%-1.0%, or 0.5%-5.0%, by weight, of all product-related impurities, process-related impurities, residual solvents, and heavy metals, derived from the (S)-(+)-flurbiprofen.

In an embodiment of this aspect of the invention there is provided a pharmaceutical composition having a component containing (S)-(+)-2-(2-fluoro-4-biphenylyl) propionic acid, or a salt or ester thereof, admixed with one or more pharmaceutically acceptable excipients, wherein the weight of the (S)-(+)-2-(2-fluoro-4-biphenylyl) propionic acid, or a salt or ester thereof, is about ≧30%, ≧35%, ≧40%, ≧45%, ≧50%, or ≧55% of the total weight of the pharmaceutical composition, and further having limited amounts of the impurities arising from the (S)-(+)-flurbiprofen, as described above. In some of these embodiments, (S)-(+)-2-(2-fluoro-4-biphenylyl) propionic acid, or a salt or ester thereof, component can be about ≧55%, ≧60%, ≧62%, ≧64%, ≧66%, ≧68% or ≧70%, of the total weight of the pharmaceutical composition. In some of these embodiments, the pharmaceutical composition is designed to contain about ≧1 mg, about ≧2 mg, about ≧3 mg, about ≧4 mg, about ≧5 mg, about ≧6 mg, about ≧7 mg, about ≧8 mg, about ≧9 mg, about ≧10 mg, about ≧15 mg, about ≧20 mg, about ≧30 mg, about ≧40 mg, about ≧50 mg or about ≧100 mg of (S)-(+)-2-(2-fluoro-4-biphenylyl) propionic acid, or a salt or ester thereof, of pharmaceutical composition.

In another aspect, the present invention provides a dosage form comprising a therapeutically effective amount of (S)-(+)-2-(2-fluoro-4-biphenylyl) propionic acid, or a salt or ester thereof, as the active ingredient with limited quantities of impurities arising from the (S)-(+)-flurbiprofen used to prepare the pharmaceutical composition used to make this dosage form. This dosage forms can be designed for oral administration, but are preferably for parenteral administration.

These dosage forms comprise pharmaceutical compositions that, in turn, comprise the drug substance preparations of the invention, which contain (S)-(+)-2-(2-fluoro-4-biphenylyl) propionic acid, or a salt or ester thereof, and limited amounts of impurities, as discussed above. In one set of embodiments, all of the impurities present in these dosage forms represent about 1%, 0.5%, 0.25% 0.1%, 0.05%, 0.025%, 0.01%, 0.005%, 0.0025%, 0.001%, or less of the total weight of the dosage form as impurities, i.e.

[sum of weight(s) of impurities derived from the drug substance preparation]×100% [total weight of dosage form]

In another embodiment, the invention provides dosage forms having from 1-0.1%, 0.1-0.01% w/w or 0.01-0.001% w/w of the all the impurities arising from the (S)-(+)-flurbiprofen, as hereinbefore described. In another embodiment, the invention provides a dosage form having from 1-0.001% w/w, 0.5-0.001% w/w, 0.25-0.001% w/w, 0.1-0.001% w/w, 0.05-0.001% w/w, 0.025-0.001% w/w or 0.01-0.001% w/w of one or more impurities arising from the (S)-(+)-flurbiprofen as hereinbefore described.

In certain embodiments of this aspect, the invention provides a unit dosage form having (S)-(+)-2-(2-fluoro-4-biphenylyl) propionic acid, or a salt or ester thereof, admixed with one or more pharmaceutically acceptable excipients, wherein the weight of (S)-(+)-2-(2-fluoro-4-biphenylyl) propionic acid, or a salt or ester thereof, is about ≧30%, about ≧35%, about ≧40%, about ≧45%, about ≧50%, about ≧55%, about ≧60%, about ≧65%, about ≧70%, about ≧75%, about ≧80%, about ≧85%, about ≧90%, about ≧95% or about ≧99% of the total weight of the unit dosage form, and further having a limited amounts of impurities arising from the (S)-(+)-flurbiprofen used to prepare the pharmaceutical composition or the unit dosage form.

Pharmaceutical compositions may contain, e.g., from about 0.1% to about 99.9%, preferably from about 20% to about 60%, of (S)-(+)-2-(2-fluoro-4-biphenylyl) propionic acid, or a salt or ester thereof, as the active ingredient. Pharmaceutical compositions may be for enteral or parenteral administration and include those, e.g. in unit dosage form, such as tablets including sugar-coated tablets, capsules, suppositories and ampoules. These are prepared in a manner known, per se, e.g., by means of conventional mixing, granulating, sugar-coating, dissolving or lyophilizing processes, etc.

The unit dosage form of these embodiments can be provided as a unit dosage form specifically suited for oral administration, e.g. a tablet, capsule or caplet, etc as hereinbefore described. This embodiment of the invention is manufactured using a pharmaceutical composition comprising (S)-(+)-2-(2-fluoro-4-biphenylyl) propionic acid, or a salt or ester thereof, that has from 30% to 90%, 35% to 90%, 40% to 90%, 45% to 90%, 50% to 90%, 55% to 90% or from 60 to 99% by weight, (S)-(+)-2-(2-fluoro-4-biphenylyl) propionic acid, or a salt or ester thereof, and from 1% to 45%, by weight, inactive pharmaceutical ingredients, and from 2%-0.001%, by weight, of the impurities arising from the (S)-(+)-flurbiprofen as hereinbefore described; wherein all of the percentages, by weight, are of the total weight of the dosage form. In a specific embodiment, the unit dosage form has from 55% to 85% by weight (S)-(+)-2-(2-fluoro-4-biphenylyl) propionic acid and 15%-45% by weight inactive pharmaceutical ingredients. In another specific embodiment, the unit dosage form has from 55% to 75% by weight (S)-(+)-2-(2-fluoro-4-biphenylyl) propionic acid and from 25% to 45% by weight inactive ingredients. In another specific embodiment, the unit dosage form has from 60% to 70% by weight (S)-(+)-2-(2-fluoro-4-biphenylyl) propionic acid and from 30% to 40% by weight inactive pharmaceutical ingredients.

In another embodiment, the invention provides a tablet, lozenge or transdermal dosage form having between about 25 and about 500 mg (S)-(+)-2-(2-fluoro-4-biphenylyl) propionic acid, or a salt or ester thereof. According to this embodiment, the dosage form also has a limited amount of impurities arising from the drug substance preparation as hereinbefore described.

In some embodiments of this aspect of the invention, each dosage form has (S)-(+)-2-(2-fluoro-4-biphenylyl) propionic acid, or a salt or ester thereof, and limited amount of impurities arising from the (S)-(+)-flurbiprofen as hereinbefore described, plus one or more binders, one or more diluents, one or more disintegrants, one or more glidants, one or more lubricants, and if desired, one or more optional ingredients. In one set of embodiments, a tablet dosage form is coated.

In a specific embodiment, the invention provides a tablet dosage form containing about 50 to 100 mg (S)-(+)-2-(2-fluoro-4-biphenylyl) propionic acid, or a salt or ester thereof, and having about 30%, 35%, 40%, 45%, 50%, or 55%, or more, by weight, of (S)-(+)-2-(2-fluoro-4-biphenylyl) propionic acid in the tablet dosage form, and limited amounts of impurities arising from the (S)-(+)-flurbiprofen as hereinbefore described, wherein the total weight of the impurities in the tablet dosage form is about 3%, 2%, 1%, 0.5%, or less of the total weight of the tablet. Furthermore, the tablet dosage forms of this embodiment are specifically suited for oral administration.

In a related embodiment, the unit dosage form is a capsule dosage form.

In one specific embodiment, the capsule dosage form comprises (S)-(+)-2-(2-fluoro-4-biphenylyl) propionic acid, or a salt or ester thereof, and limited amounts of impurities arising from the (S)-(+)-flurbiprofen as hereinbefore described used to make the pharmaceutical composition that goes into the capsules, and one or more pharmaceutically acceptable excipients, as described above, as additional components. In one set of embodiments, the capsule dosage form comprises a hard gelatin capsule that contains a pharmaceutical composition of the invention.

In a related set of embodiments, the unit dosage form is a caplet dosage form.

In another embodiment of the present invention the unit dosage form may be provided as a unit dosage form specifically suited for parenteral administration, e.g. topical or transdermal administration, such as a patch. Such patch preparations may comprise the patches described in European Patent application No. 0713697 (Tokuhon) which is incorporated herein by reference.

A pharmaceutical composition according to any one of claims 2 to 8 wherein the composition comprises an adhesive preparation obtained by coating one surface of support with an adhesive which contains (S)-(+)-2-(2-fluoro-4-biphenylyl) propionic acid, or a salt or ester thereof, and a plaster base. The plaster base may be, for example, a styrene-isoprene-styrene block copolymer or a modified copolymer in which methylmethacrylate has been allowed to graft-polymerize on said copolymer.

The adhesive may further contain a rosin ester resin as a tackifier and/or mentha oil and at least one compound selected from the group comprising intermediate chain fatty acid esters of polyhydric alcohol as a promoter for percutaneous absorption of a pharmacologically effective component.

The analgesic anti-inflammatory adhesive preparation may be obtained by coating one surface of a support with an adhesive which contains (5)-(+)-2-(2-fluoro-4-biphenylyl) propionic acid, or a salt or ester thereof, as hereinbefore described, and a plaster base, wherein the plaster base comprising sodium carboxymethyl cellulose and sodium polyacrylate.

The adhesive may further contain an emulsion of polyacrylate esters and/or mentha oil and at least one compound selected from a group consisting of intermediate chain fatty acid esters of polyhydric alcohols.

Synthetic rubbers such as butyl rubber, polyisobutylene, styrene-butadiene rubber and acrylic resins may be used as this kind of plaster base. An alkyl (meth)acrylate copolymer obtained from aliphatic alcohol with a carbon number of 4-18 and (meth)acrylic acid as well as a copolymer of the aforementioned alkyl (meth)acrylate and another functional monomer are preferable as this acrylic resin. In addition, to give specific examples of the alkyl (meth) acrylate described above, the following may be cited: butyl acrylate, isobutyl acrylate, hexyl acrylate, octyl acrylate, 2-ethylhexyl acrylate, isooctyl acrylate, decyl acrylate, isodecyl acrylate, lauryl acrylate, stearyl acrylate, methyl methacrylate, ethyl methacrylate, butyl methacrylate, isobutyl methacrylate, 2-ethylhexyl methacrylate, isooctyl methacrylate, decyl methacrylate, isodecyl methacrylate, lauryl methacrylate, and stearyl methacrylate, etc.

According to this aspect of the invention there is provided a patch for topical or transdermal administration comprising a pharmaceutical composition according to any one of claims 2 to 8, such that the (S)-(+)-2-(2-fluoro-4-biphenylyl) propionic acid, or a salt or ester thereof, is present on an adhesive support in a density of from about 50 to about 1000 μm/cm². The patch for topical or transdermal administration of (S)-(+)-2-(2-fluoro-4-biphenylyl) propionic acid, or a salt or ester thereof, will comprise the a pharmacologically effective amount of (S)-(+)-2-(2-fluoro-4-biphenylyl) propionic acid, or a salt or ester thereof, such that the (S)-(+)-2-(2-fluoro-4-biphenylyl) propionic acid, or a salt or ester thereof, is normally present on the support in a density of from about 50 to about 1000 μm/cm², preferably from about 70 to about 1000 μm/cm².

In a further embodiment of the present invention the unit dosage form may be provided as a unit dosage form specifically suited for oral administration, e.g. a lozenge. Such lozenge preparations may comprise those described in U.S. Pat. No. 6,166,083 which is incorporated herein by reference. The term “lozenge” as used herein is intended to embrace all dosage forms where the product is formed by cooling a sugar-based or sugar alcohol based (e.g. sorbitol) molten mass containing the active material. A preferred pharmaceutical composition is a lozenge prepared by cooling a heated lozenge base comprising sugar, liquid glucose, ((S)-(+)-2-(2-fluoro-4-biphenylyl) propionic acid, or a salt or ester thereof, as hereinbefore described, and other excipients to form solid lozenges. Such lozenges may be suitable for the treatment or alleviation of, inter alia, sore throats.

The therapeutically effective amount of ((S)-(+)-2-(2-fluoro-4-biphenylyl) propionic acid, or a salt or ester thereof, may be from 5% to 40% of the normal adult dose when given by ingestion to achieve a systemic anti-inflammatory and/or analgesic effect. (S)-(+)-2-(2-Fluoro-4-biphenylyl) propionic acid, or a salt or ester thereof, may therefore be present in a lozenge composition in an amount from 2.5 to 20 mg preferably 5 to 12.5 mg, Where a pharmaceutically acceptable salt of (S)-(+)-2-(2-fluoro-4-biphenylyl) propionic acid is used, the amount of the salt used should be such as to provide the desired amount of (S)-(+)-2-(2-fluoro-4-biphenylyl) propionic acid. Suitable salts for use in a lozenge composition include the salts hereinafter described, such as, the sodium salt or amino acid salts. Preferred salts include, e.g. the lysine, arginine or meglumine salts.

In a yet further embodiment of the present invention the unit dosage form may be provided as a unit dosage form specifically suited for oral administration, e.g. a syrup or suspension. Such a syrup or suspension may use a plasticizer composed of concentrated glycerin and polyoxyl 40-hardened castor oil, and the like. Such a syrup may be especially suitable for administration to children. Therefore, the syrup may also include a flavouring agent, sweetener, colouring agent and/or preservative, such as cyclodextrin liquid, disorbitol liquid, aspartame and stevioside.

The syrup or suspension according to this aspect of the invention may comprise (S)-(+)-2-(2-fluoro-4-biphenylyl) propionic acid, or a salt or ester thereof, having an average particle size ranging from 10 to 300 μm as an active ingredient, said composition having a viscosity ranging from 500 to 3,000 cps and pH ranging from 3.0 to 6.0.

The (S)-(+)-2-(2-fluoro-4-biphenylyl) propionic acid, or a salt or ester thereof, may be present in an amount ranging from 0.01 to 10.0 w/v %, preferably 0.7 to 5.0 w/v % based on the total volume of the syrup/suspension composition. The syrup or suspension may include a viscosity controlling agent in order to control the viscosity of the composition in the range from 500 to 3,000 cps. Such a viscosity controlling agent may be selected from the group consisting of agar, sodium alginate, povidone, polyethylene glycol, hydroxyethylene cellulose, D-sorbitol solution and a mixture thereof. The viscosity controlling agent acts to prevent layer separation of the syrup/suspension composition, and provides proper fluidity for oral administration, e.g. to children. The viscosity controlling agent may be employed in an amount ranging from 0.01 to 40.0 w/v %, preferably 0.1 to 30.0 w/v % based on the total volume of the syrup composition.

In summary, the invention provides (S)-(+)-2-(2-fluoro-4-biphenylyl) propionic acid, or a salt or ester thereof, a drug substance preparation, a pharmaceutical composition or dosage form containing (S)-(+)-2-(2-fluoro-4-biphenylyl) propionic acid, or a salt or ester thereof, as the active ingredient, and about 5%, 4%, 3%, 2%, 1%, 0.5%, 0.25%, 0.2%, 0.15%, 0.1%, 0.01% or less (of the total weight of a particular composition) of all of the product-related impurities, process-related impurities, residual solvents and heavy metals, as hereinbefore defined.

In another embodiment, the invention provides (S)-(+)-2-(2-fluoro-4-biphenylyl) propionic acid, or a salt or ester thereof, a drug substance preparation, pharmaceutical composition, or dosage form containing (S)-(+)-2-(2-fluoro-4-biphenylyl) propionic acid, or a salt or ester thereof, as the active ingredient, and about 0.005%, 0.01%, 0.03%, 0.05%, 0.07%, 0.08%, 0.09% or 0.1% or more (of the total weight of a particular composition) of one or more of the product-related impurities, process-related impurities, residual solvents and heavy metals, as hereinbefore defined.

In a specific embodiment, the invention provides (S)-(+)-2-(2-fluoro-4-biphenylyl) propionic acid, or a salt or ester thereof, a drug substance preparation, pharmaceutical composition, or dosage form having from 5-0.5%, 1-0.1%, 0.5-0.05%, 0.1-0.01%, 0.05-0.005%, or 0.01-0.001% of one or more of the product-related impurities, process-related impurities, residual solvents and heavy metals, as defined below. In another specific embodiment, the invention provides a drug substance preparation, pharmaceutical composition, or dosage form having from 5-0.5%, 1-0.1%, 0.5-0.05%, 0.1-0.01%, 0.05-0.005%, or 0.01-0.001% of all of the product-related impurities, process-related impurities, residual solvents and heavy metals, as defined below.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, examples of suitable methods and materials are described below. In case of conflict, the present specification, including definitions, will control. In addition, the materials, methods, and examples are illustrative only and are not intended to be limiting.

Other features and advantages of the invention will be apparent from the following detailed description, and from the claims.

The drug substance preparations, pharmaceutical compositions, and dosage forms of the invention as hereinbefore described all contain (S)-(+)-2-(2-fluoro-4-biphenylyl) propionic acid, or a salt or ester thereof, as the active ingredient.

(S)-(+)-2-(2-fluoro-4-biphenylyl) propionic acid is the “S” enantiomer of flurbiprofen ((R,S)-2-(2-fluoro-4-biphenylyl) propionic acid). (S)-(+)-2-(2-fluoro-4-biphenylyl) propionic acid can be obtained by resolving racemic flurbiprofen or non-racemic mixtures of (R)-(−)-flurbiprofen and (S)-(+)-flurbiprofen into its separate enantiomeric forms, or through enantioselective or enantiospecific syntheses.

The S-enantiomer of flurbiprofen ((S)-(+)-2-(2-fluoro-4-biphenylyl) propionic acid), or a particular desired enantiomeric excess of (S)-(+)-2-(2-fluoro-4-biphenylyl) propionic acid, can be obtained by resolving the racemic flurbiprofen or non-racemic mixtures of (R)-(−)-flurbiprofen and (S)-(+)-flurbiprofen according to known methods, as noted herein.

The S-enantiomer of flurbiprofen ((S)-(+)-2-(2-fluoro-4-biphenylyl) propionic acid), is commercially available. However, the identities and amounts of product-related and process-related impurities found in preparations of (S)-(+)-2-(2-fluoro-4-biphenylyl) propionic acid, have never been described, and never held to specifically-defined limits.

The term “(S)-(+)-2-(2-fluoro-4-biphenylyl) propionic acid,” as used herein, refers to the free acid form of (S)-(+)-2-(2-fluoro-4-biphenylyl) propionic acid, and to salts and esters thereof.

Where salts and/or esters are employed, they will usually comprise pharmaceutically acceptable salts and/or esters. The specific amounts and ranges of salts to be used are ideally the amounts and ranges that are bioequivalent to those indicated for the free acid. That is to say, if a pharmaceutically acceptable salt is used in a drug substance preparation, composition, dosage form, or drug product of the invention, it should be used in the amount necessary to provide a therapeutic effect equivalent to that obtained with the free acid form of (S)-(+)-2-(2-fluoro-4-biphenylyl) propionic acid (i.e. a “therapeutically equivalent” amount of the pharmaceutically acceptable salt or ester). In most instances, this simply means using a molar equivalent amount of the pharmaceutically acceptable salt or ester in place of the specified amount of the free acid form used in the particular embodiment.

As used herein, the term “dose” or “dosage” refers to the amount of active pharmaceutical ingredient that an individual takes or is administered at one time. For example, a 100 mg (S)-(+)-2-(2-fluoro-4-biphenylyl) propionic acid dose refers to, in the case of a twice-daily dosage regimen, a situation where, for example, the individual takes, or is administered, 100 mg (S)-(+)-2-(2-fluoro-4-biphenylyl) propionic acid in the morning and 100 mg (S)-(+)-2-(2-fluoro-4-biphenylyl) propionic acid in the evening. The 100 mg (S)-(+)-2-(2-fluoro-4-biphenylyl) propionic acid dose can be a single unit or can be divided into two or more dosage units, e.g., two 50 mg (S)-(+)-2-(2-fluoro-4-biphenylyl) propionic acid tablets or two 50 mg (S)-(+)-2-(2-fluoro-4-biphenylyl) propionic acid capsules. As used herein, the term “unit dosage form” refers to a physically discrete unit, such as a tablet or capsule suitable as a unitary dosage for a human patient. Each unit contains a predetermined quantity of (S)-(+)-2-(2-fluoro-4-biphenylyl) propionic acid that was discovered to produce a desired pharmacokinetic profile which yields the desired therapeutic effect.

The term “active pharmaceutical ingredient,” or “active ingredient,” as used herein in the context of the drug substance preparations, pharmaceutical compositions, and dosage forms or drug products of the invention, refers to the free acid form of (S)-(+)-2-(2-fluoro-4-biphenylyl) propionic acid,

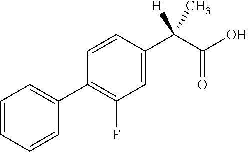 [SMILES: C[C@H](C(=O)O)c1ccc(-c2ccccc2)c(F)c1]

and to pharmaceutically acceptable salt forms thereof

As used herein, the terms “drug substance” and “drug substance preparation,” refers to the active ingredient-containing material that is used to formulate, along with excipients, the pharmaceutical compositions, dosage forms, and drug products of the invention. It is composed of the active ingredient, and limited quantities of specific product-related impurities, process-related impurities, residual solvents, and heavy metals.

The term “excipient,” as used herein, refers to those components of a pharmaceutical composition, dosage form, or drug product, other than the drug substance, that are intentionally included in the composition or formulation to either facilitate manufacture, enhance stability, control the release of the active ingredient from the drug product, assist in product identification, or enhance any other product characteristics, including, for example, the pharmacokinetics of the drug product. Generally, excipients may be thought of as the “inactive ingredients” of the pharmaceutical composition, dosage form, or drug product, in the sense that they exert no direct therapeutic effect. However, excipients can have a significant effect on the pharmacokinetic characteristics of pharmaceutical compositions, dosage forms, or drug products containing the active ingredient, by influencing such parameters as dissolution, and release of the active ingredient.

As used herein, the term “pharmaceutical composition” is used to refer to compositions of matter comprising the drug substance and one or more pharmaceutically acceptable excipients. Additionally, these terms are meant to refer to compositions of matter (containing the drug substance and one or more excipients) that are used to prepare drug products or dosage forms, along with one or more additional excipients.

As used herein, the terms “drug product,” “dosage form,” or “finished product” are used interchangeably to refer to a finished pharmaceutical product or medicament that is suitable for administration to a human patient. The drug product or dosage form comprises the drug substance and pharmaceutically acceptable excipients, and can also be thought of as comprising a pharmaceutical composition in combination with one or more additional excipients. One example of a drug product or dosage form is a “tablet dosage form,” or “tablet,” which is formulated and manufactured for the gastrointestinal administration of the active ingredient by an oral route (i.e. oral administration).

The term “impurity,” as used herein, refers to any component present in the drug substance (or a drug substance preparation), a pharmaceutical composition thereof, or a drug product or dosage form thereof, that is neither the active ingredient nor an excipient. For the sake of this application, the term “impurity” generally refers to impurities arising from, or contained in, the drug substance, or a drug substance preparation. Consequently, the term “impurity” comprises product-related impurities, process-related impurities, residual solvents, and heavy metals, which are found in the drug substance or drug substance preparations.

As used herein, the term “product-related impurities” refers to organic chemicals that are structurally similar to the active ingredient, that are found in drug substance preparations, but that do not have properties comparable to those of the active ingredient with respect to activity, efficacy, and safety. Product-related impurities can also include “degradants,” which are products of the degradation of the active ingredient. The term “product-related impurities” also encompasses enantiomeric impurities. In the instant invention the sole enantiomeric impurity is:

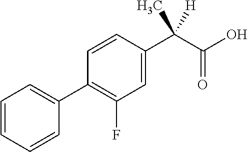 [SMILES: C[C@@H](C(=O)O)c1ccc(-c2ccccc2)c(F)c1]

(R)-(−)-2-(2-fluoro-4-biphenylyl) propionic acid;
   which is also known as (R)-(−)-flurbiprofen. However, given that this enantiomeric impurity is of particular concern in the drug substance preparations, pharmaceutical compositions, and dosage forms of the invention, it will generally be considered apart from, or in addition to, all other product-related impurities.

Specific examples of “product-related impurities” besides (R)-(−)-2-(2-fluoro-4-biphenylyl) propionic acid found in the drug substance preparations, pharmaceutical compositions and dosage forms of the instant invention, can include:

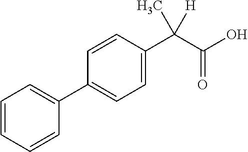 [SMILES: CC(C(=O)O)c1ccc(-c2ccccc2)cc1]

2-(4-biphenylyl) propionic acid;

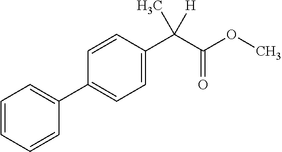 [SMILES: COC(=O)C(C)c1ccc(-c2ccccc2)cc1]

methyl(2-(2-fluoro-4-biphenylyl)) propionate;

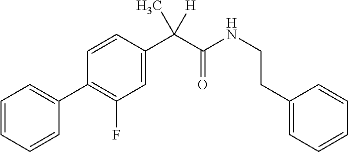 [SMILES: CC(C(=O)NCCc1ccccc1)c1ccc(-c2ccccc2)c(F)c1]

1-phenylethyl-(2-2(fluorobiphenyl-4-yl)) propionamide; and

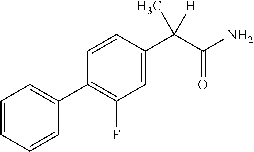 [SMILES: CC(C(N)=O)c1ccc(-c2ccccc2)c(F)c1]

2-(2-fluorobiphenyl-4-yl) propionamide;
   and their chiral counterparts where appropriate.

The term “process-related impurities,” as used herein, refers to impurities other than product-related impurities, which derive from the process used to manufacture the drug substance preparation. Process-related impurities commonly found in drug substance preparations include residual solvents, catalysts and other compounds used in the synthesis of the active ingredient, heavy metals, and compounds used during the purification the active ingredient.

Specific examples of “process-related impurities” in the instant invention include the solvents toluene, methanol and n-heptane, heavy metals (measured as ppm Pb), and the chiral crystallization agent (S)-(−)-α-methylbenzylamine, which is also known as (S)-(−)-1-phenylethylamine.

All drug substance preparations in which the active ingredient is a small organic molecule (such as (S)-(+)-2-(2-fluoro-4-biphenylyl) propionic acid) that is synthesized from smaller, more readily-available starting materials, and isolated from organic solvents, can be expected to contain impurities. Generally, the types of impurities seen in such drug substance preparations are dictated by the synthetic route and specific processes used to prepare the active ingredient. Certainly, the amounts of impurities observed in drug substance preparations can be reduced or controlled by steps taken at various points during the synthesis and purification of the active ingredient.

In the present invention, drug substance preparations are provided that contain (S)-(+)-2-(2-fluoro-4-biphenylyl) propionic acid, or a pharmaceutically acceptable salt thereof, as the active ingredient. These drug substance preparations are made using the methods provided that substantially reduce the amounts of impurities present, and limit the amounts impurities that remain. Hence, the drug substance preparations of the present invention contain limited amount of specific impurities, the identity and quantity of which have been determined, and are described herein.

As noted above, impurities that occur in the (S)-(+)-2-(2-fluoro-4-biphenylyl) propionic acid-containing drug substance preparations of the present invention include product-related impurities, process-related impurities, residual solvents and heavy metals. Product-related impurities known to occur within the drug substance preparations of the present invention include the enantiomeric impurity (R)-(−)-2-(2-fluoro-4-biphenylyl) propionic acid (i.e., (R-flurbiprofen), as well as 2-(4-biphenylyl) propionic acid and methyl(2-(2-fluoro-4-biphenylyl)) propionate, and occasionally include 1-phenylethyl-(2-2(fluorobiphenyl-4-yl)) propionamide and 2-(2-fluorobiphenyl-4-yl) propionamide. Process-related impurities known to occur within the drug substance preparations of the present invention include the chiral crystallization agent (S)-(−)-α-methylbenzylamine, as well as residual solvents, including toluene, methanol and n-heptane, and trace amounts of heavy metals.

In view of the above, a first aspect of the present invention provides drug substance preparations containing (S)-(+)-2-(2-fluoro-4-biphenylyl) propionic acid, or a salt or ester thereof, and limited amounts of specific product-related impurities, process-related impurities, residual solvents and heavy metals. In one embodiment of this aspect, all of the impurities present in these drug substance preparations are limited to about 5%, 4%, 3%, 2%, 1%, or less of the total weight of the drug substance preparation, i.e.

[sum of weight(s) of one or more impurities]×100% [total weight of drug substance preparation]

is less than about 5%, 4%, 3%, 2%, 1%, or less).

In another embodiment, the invention provides a drug substance preparation containing (S)-(+)-2-(2-fluoro-4-biphenylyl) propionic acid, or a salt or ester thereof, and about 2%, 1%, 0.5%, 0.25%, 0.1%, 0.05%, 0.025%, 0.01%, 0.005%, 0.0025%, or 0.001% of the total weight of the drug substance preparation as product-related impurities.

In another embodiment, the invention provides a drug substance preparation having from 0.001%-0.01%, 0.005%-0.05%, 0.01%-0.1%, 0.05%-0.5%, or 0.1%-1%, of any one specific impurity, by weight. In another embodiment, the invention provides a drug substance preparation having from 0.001%-0.01%, 0.005%-0.05%, 0.01%-0.1%, 0.05%-0.5%, 0.1%-1%, or 0.5%-5%, by weight, of the specific product-related impurities, process-related impurities, residual solvents and heavy metals identified herein.

In certain embodiments, the present invention comprises drug substance preparations containing (S)-(+)-2-(2-fluoro-4-biphenylyl) propionic acid as the active ingredient, and limited amounts of product-related impurities. In these embodiments, the product-related impurities that are present in limited amounts include the enantiomeric impurity (R)-(−)-2-(2-fluoro-4-biphenylyl) propionic acid, which is also known as (R)-flurbiprofen, 2-(4-biphenylyl) propionic acid, and methyl(2-(2-fluoro-4-biphenylyl))propionate. In specific embodiments, the present invention comprises drug substance preparations wherein the amounts of 2-(4-biphenylyl) propionic acid, as well as the enantiomeric impurity (R)-(−)-2-(2-fluoro-4-biphenylyl) propionic acid, are limited below certain maximum levels. In other specific embodiments, the present invention comprises drug substance preparations wherein the amounts of both 2-(4-biphenylyl) propionic acid and methyl(2-(2-fluoro-4-biphenylyl))propionate, as well as the enantiomeric impurity (R)-(−)-2-(2-fluoro-4-biphenylyl) propionic acid, are limited below certain maximum levels.

In certain embodiments, the present invention comprises drug substance preparations wherein, in addition to the amounts of one or two known product-related impurities, as well as the enantiomeric impurity (R)-(−)-2-(2-fluoro-4-biphenylyl) propionic acid, being limited below certain maximum levels, the amounts of specific process-related impurities are also limited below certain maximum levels. In these embodiments, the principle process-related impurity that is limited below certain maximum levels is (S)-(−)-α-methylbenzylamine, which is also known as (R)-(+)-1-phenylethylamine, and which is used to resolve and isolate (S)-(+)-2-(2-fluoro-4-biphenylyl) propionic acid from racemic flurbiprofen (i.e., (R,S)-2-(2-fluoro-4-biphenylyl) propionic acid) or non-racemic mixtures of (R)-(−)-flurbiprofen and (S)-(+)-flurbiprofen. In further embodiments, additional process-related impurities that are limited below certain maximum levels include residual solvents, and/or heavy metals. In these embodiments, the residual solvents to be limited include toluene, methanol, and n-heptane.

In a preferred embodiment of this aspect of the invention, the present invention comprises drug substance preparations containing: (S)-(+)-2-(2-fluoro-4-biphenylyl) propionic acid as the active ingredient; limited amounts of three product-related impurities, including the enantiomeric impurity (R)-(−)-2-(2-fluoro-4-biphenylyl) propionic acid, 2-(4-biphenylyl) propionic acid, and methyl(2-(2-fluoro-4-biphenylyl))propionate; limited amounts of the process-related impurity (S)-(−)-α-methylbenzylamine; and limited amounts of residual solvents and heavy metals.

As demonstrated, select product-related impurities detected in an exemplary batch of (S)-(+)-2-(2-fluoro-4-biphenylyl) propionic acid-containing drug substance preparations include, for example, 2-(4-biphenylyl) propionic acid and methyl(2-(2-fluoro-4-biphenylyl)) propionate, in addition to the R-enantiomer of flurbiprofen, or (R)-(−)-2-(2-fluoro-4-biphenylyl) propionic acid. Similarly, process-related impurities detected in an exemplary batch of (S)-(+)-2-(2-fluoro-4-biphenylyl) propionic acid-containing drug substance preparations include, for example, (S)-(−)-α-methylbenzylamine, residual toluene, residual methanol, and residual n-heptane, as well as trace amounts of heavy metals. Importantly, the inventive drug substance preparations containing limited amount of the above-listed impurities allow for the production of pharmaceutical compositions and dosage forms having therapeutically effective amounts of (S)-(+)-2-(2-fluoro-4-biphenylyl) propionic acid (or therapeutically equivalent amounts of a pharmaceutically acceptable salt thereof), but substantially limited quantities of impurities.

Thus, the inventors have discovered methods of manufacturing (S)-(+)-2-(2-fluoro-4-biphenylyl) propionic acid-containing drug substance that contains substantially limited amounts of specific impurities, which is subsequently used to prepare the pharmaceutical compositions, and, ultimately, the dosage forms of the present invention.

Therefore, according to a yet further aspect of the invention there is provided a process for the preparation of (S)-(+)-2-(2-fluoro-4-biphenylyl) propionic acid, or a salt or ester thereof, and having substantially limited amounts of specific impurities associated with the synthesis and purification of the (S)-(+)-2-(2-fluoro-4-biphenylyl) propionic acid, said process comprising:
(i) reacting a solution of flurbiprofen and (S)-(−)-α-alkyl(C₁₋₆)benzylamine;
(ii) isolating (S)-(+)-flurbiprofen-(S)-(−)-α-alkyl(C₁₋₆)benzylamine formed in step (i);
(iii) optionally recrystallising the (S)-(+)-flurbiprofen-(S)-(−)-α-alkyl(C₁₋₆)benzylamine;
(iv) reacting an organic solution of (S)-(+)-flurbiprofen-(S)-(−)-α-alkyl(C₁₋₆)benzylamine with an acid to form (S)-(+)-flurbiprofen;
(v) separating the organic portion containing (S)-(+)-flurbiprofen;
(vi) optionally washing the organic portion with a further acid; and
(vii) isolating the crystalline (S)-(+)-flurbiprofen.

It will be understood by the person skilled in the art that;
the flurbiprofen used in step (i) may be racemic flurbiprofen, although other non-racemic mixtures of (R)-(−)-flurbiprofen and (S)-(+)-flurbiprofen may suitably be used; and
the acids used in steps (iv) and (vi) above may be the same or different.

The (S)-(−)-α-alkyl(C₁₋₆)benzylamine used in the process described herein is preferably (S)-(−)-α-methylbenzylamine.

When these impurities are considered separately in the context of drug substance preparations, one arrives at the following embodiments of this aspect of the invention.

In some embodiments, the (S)-(+)-2-(2-fluoro-4-biphenylyl) propionic acid-containing drug substance preparations used in the pharmaceutical compositions and dosage forms of the invention has less than about 1.0%, 0.9%, 0.8%, 0.7%, 0.6%, 0.5%, 0.4%, 0.3%, 0.2%, or 0.1%, by weight, 2-(4-biphenylyl) propionic acid.

In some embodiments, the (S)-(+)-2-(2-fluoro-4-biphenylyl) propionic acid-containing drug substance preparations used in the pharmaceutical compositions and dosage forms of the invention has less than about 0.2%, 0.18%, 0.16%, 0.14%, 0.12%, 0.1%, 0.09%, 0.08%, 0.07%, or 0.06%, by weight, methyl(2-(2-fluoro-4-biphenylyl))propionate.

In some embodiments, the (S)-(+)-2-(2-fluoro-4-biphenylyl) propionic acid-containing drug substance preparations used in the pharmaceutical compositions and dosage forms of the invention has less than about 0.2%, 0.18%, 0.16%, 0.14%, 0.12%, 0.1%, 0.09%, 0.08%, 0.07%, or 0.06%, by weight, 1-phenylethyl-(2-2(fluorobiphenyl-4-yl)) propionamide.

In some embodiments, the (S)-(+)-2-(2-fluoro-4-biphenylyl) propionic acid-containing drug substance preparations used in the pharmaceutical compositions and dosage forms of the invention has less than about 0.2%, 0.18%, 0.16%, 0.14%, 0.12%, 0.1%, 0.09%, 0.08%, 0.07%, or 0.06%, by weight, 2-(2-fluorobiphenyl-4-yl) propionamide.

In some embodiments, the (S)-(+)-2-(2-fluoro-4-biphenylyl) propionic acid-containing drug substance preparations used in the pharmaceutical compositions and dosage forms of the invention has an enantiomeric excess of (S)-(+)-2-(2-fluoro-4-biphenylyl) propionic acid of about 95%, 98%, 99%, 99.1%, 99.2%, 99.3%, 99.4%, 99.5%, 99.6%, 99.7%, 99.8%, or 99.9% over the R-enantiomer of flurbiprofen, namely (R)-(−)-2-(2-fluoro-4-biphenylyl) propionic acid. Respectively, in these embodiments, the (S)-(+)-2-(2-fluoro-4-biphenylyl) propionic acid-containing drug substance preparations used in the pharmaceutical compositions and dosage forms of the invention has not more than about 3%, 2%, 1%, 0.5%, 0.45%, 0.4%, 0.35%, 0.3%, 0.25%, 0.2%, 0.15%, 0.1%, or 0.05%, or less, by weight, (R)-(−)-2-(2-fluoro-4-biphenylyl) propionic acid.

In some embodiments, the (S)-(+)-2-(2-fluoro-4-biphenylyl) propionic acid-containing drug substance preparations used in the pharmaceutical compositions and dosage forms of the invention has not more than about 1000 ppm, 800 ppm, 600 ppm, 500 ppm, 400 ppm, 300 ppm, 250 ppm, 200 ppm, 100 ppm, 50 ppm, 25 ppm, 10 ppm, or 5 ppm, or less, of the process-related impurity, (S)-(−)-α-methylbenzylamine.

In some embodiments, the (S)-(+)-2-(2-fluoro-4-biphenylyl) propionic acid-containing drug substance preparations used in the pharmaceutical compositions and dosage forms of the invention has less than about 900 ppm, 800 ppm, 700 ppm, 600 ppm, 500 ppm, 400 ppm, 300 ppm, 200 ppm, or 100 ppm toluene.

In some embodiments, the (S)-(+)-2-(2-fluoro-4-biphenylyl) propionic acid-containing drug substance preparations used in the pharmaceutical compositions and dosage forms of the invention has less than about 0.5%, 0.4%, 0.3%, 0.2%, 0.1%, 0.05%, or 0.025%, by weight, methanol.

In some embodiments, the (S)-(+)-2-(2-fluoro-4-biphenylyl) propionic acid-containing drug substance preparations used in the pharmaceutical compositions and dosage forms of the invention has less than about 0.5%, 0.4%, 0.3%, 0.2%, 0.1%, 0.05%, or 0.025%, by weight, n-heptane.

In some embodiments, the (S)-(+)-2-(2-fluoro-4-biphenylyl) propionic acid acid-containing drug substance preparations used in the pharmaceutical compositions and dosage forms of the invention has less than about 100 ppm, 50 ppm, 25 ppm, 10 ppm, 8 ppm, 6 ppm, 4 ppm, 2 ppm, or 1 ppm heavy metals (calculated as Pb), as determined by the Ph Eur, USP method.

In light of the above, the invention provides drug substance preparations comprising (S)-(+)-2-(2-fluoro-4-biphenylyl) propionic acid, or a pharmaceutically acceptable salt thereof, as the active pharmaceutical ingredient, and one or more of the above-identified impurities, in the above-identified amounts.

In a preferred embodiment, the invention provides a drug substance preparations comprising (S)-(+)-2-(2-fluoro-4-biphenylyl) propionic acid, or a pharmaceutically acceptable salt thereof, as the active pharmaceutical ingredient, and between about 0.001% and about 3%, by weight, product-related impurities, wherein said product-related impurities comprise not more than about 0.5%, by weight, 2-(4-biphenylyl) propionic acid and not more than 2%, by weight, of the enantiomeric impurity (R)-(−)-2-(2-fluoro-4-biphenylyl) propionic acid, or a salt thereof.

In another preferred embodiment, the invention provides a drug substance preparation comprising:

(1) (S)-(+)-2-(2-fluoro-4-biphenylyl) propionic acid, or a pharmaceutically acceptable salt thereof, as the active pharmaceutical ingredient;

(2) between about 0.001% and about 2%, by weight, product-related impurities, wherein said product-related impurities comprise:
(a) not more than about 0.5%, by weight, 2-(4-biphenylyl) propionic acid;
(b) not more than about 0.1%, by weight, methyl(2-(2-fluoro-4-biphenylyl)) propionate, and
(c) not more than about 1.0%, by weight, of the enantiomeric impurity (R)-(−)-2-(2-fluoro-4-biphenylyl) propionic acid, or a salt thereof;

(3) not more than about 50 ppm of the process-related impurity, (S)-(−)-α-methylbenzylamine;

(4) not more than about 900 ppm of toluene, not more than about 0.3%, by weight, methanol, and not more than about 0.3%, by weight, n-heptane; and

(5) not more than about 10 ppm heavy metals.

Additionally, in certain embodiments, the (S)-(+)-2-(2-fluoro-4-biphenylyl) propionic acid-containing drug substance preparations used in the pharmaceutical compositions and dosage forms of the invention has less than about 0.2%, 0.18%, 0.16%, 0.14%, 0.12%, 0.1%, 0.09%, 0.08%, 0.07%, or 0.06%, by weight, of any other specifically named impurity.

In other embodiments, the (S)-(+)-2-(2-fluoro-4-biphenylyl) propionic acid-containing drug substance preparations used in the pharmaceutical compositions and dosage forms of the invention has less than about 0.2%, 0.18%, 0.16%, 0.14%, 0.12%, 0.1%, 0.09%, 0.08%, 0.07%, or 0.06%, by weight, unidentified impurities.

In other embodiments, the (S)-(+)-2-(2-fluoro-4-biphenylyl) propionic acid-containing drug substance preparations used in the pharmaceutical compositions and dosage forms of the invention has no detectable impurities. Alternatively, the impurities are detectable but are less than about 0.01% of the weight of the drug substance preparation.

In other embodiments, the (S)-(+)-2-(2-fluoro-4-biphenylyl) propionic acid-containing drug substance preparations used in the pharmaceutical compositions and dosage forms of the invention has less than about 0.2%, 0.18%, 0.16%, 0.14%, 0.12%, 0.1%, 0.09%, 0.08%, 0.07%, or 0.06%, by weight, residue upon ignition.

In other embodiments, the (S)-(+)-2-(2-fluoro-4-biphenylyl) propionic acid acid-containing drug substance preparations used in the pharmaceutical compositions and dosage forms of the invention has less than about 1.0%, 0.9%, 0.8%, 0.7%, 0.6%, 0.5%, 0.4%, 0.3%, 0.2%, or 0.1% loss of weight on drying for 3 or more hours at 60° C. with pressure not exceeding 5 mm of Hg over phosphorous pentoxide.

(S)-(+)-2-(2-fluoro-4-biphenylyl) propionic acid, or a salt or ester thereof is a non-steroidal anti-inflammatory drug (NSAID) and is a pharmaceutically active agent which is therapeutically useful as in the treatment or alleviation of, inter alia, inflammation and pain.

Therefore, according a to a yet further aspect of the invention there is provided a method of treatment or alleviation of inflammation and/or pain which comprises administering a therapeutically effective amount of (S)-(+)-2-(2-fluoro-4-biphenylyl) propionic acid, or a salt or ester thereof, having substantially limited amounts of specific impurities associated with the synthesis and purification of the (S)-(+)-2-(2-fluoro-4-biphenylyl) propionic acid, to a patient in need of such treatment.

The invention further provides the use of (S)-(+)-2-(2-fluoro-4-biphenylyl) propionic acid, or a salt or ester thereof, having substantially limited amounts of specific impurities associated with the synthesis and purification of the (S)-(+)-2-(2-fluoro-4-biphenylyl) propionic acid, in the manufacture of a medicament, e.g. a medicament for the treatment or alleviation of inflammation and/or pain.

In another aspect, the present invention provides pharmaceutical compositions comprising the drug substance preparations hereinbefore described.

As described above, (S)-(+)-2-(2-fluoro-4-biphenylyl) propionic acid, or a salt or ester thereof, containing drug substance preparations include specific product-related impurities, process-related impurities, residual solvents and trace amounts of heavy metals. Since these drug substance preparations are used to prepare the pharmaceutical compositions of the invention, the pharmaceutical compositions of the invention also include the product-related impurities, 2-(4-biphenylyl) propionic acid and methyl(2-(2-fluoro-4-biphenylyl)) propionate, in addition to the R-enantiomer of flurbiprofen, namely (R)-(−)-2-(2-fluoro-4-biphenylyl) propionic acid. Similarly, the pharmaceutical compositions of the invention also include the process-related impurities such as, for example, (S)-(−)-α-methylbenzylamine, residual toluene, residual methanol, and residual n-heptane, as well as trace amounts of heavy metals.

Consequently, in embodiments of this aspect of the invention, the pharmaceutical compositions of the invention comprise (S)-(+)-2-(2-fluoro-4-biphenylyl) propionic acid as the active ingredient, and further comprise limited quantities of product-related impurities, process-related impurities, residual solvents and heavy metals. In these embodiments, the present invention provides pharmaceutical compositions in which all of the impurities deriving from the drug substance preparations of the invention represent about 2%, 1%, 0.5%, 0.25% 0.1%, 0.05%, 0.025%, 0.01%, or less of the total weight of the pharmaceutical composition, i.e.

[sum of weight(s) of impurities deriving from the drug substance preparation]×100% [total weight of pharmaceutical composition]

In certain embodiments of this aspect, the invention provides pharmaceutical compositions containing (S)-(+)-2-(2-fluoro-4-biphenylyl) propionic acid, or a salt or ester thereof, as the active ingredient, and about 0.5%, 0.25%, 0.1%, 0.05%, 0.025%, 0.01%, or 0.005% or less of the total weight of the pharmaceutical compositions as impurities arising from the drug substance preparation used to prepare the composition. In other embodiments, the invention provides a pharmaceutical composition having from 0.001%-0.01%, 0.01%-0.1%, or 0.1%-1% of one or more impurities, by weight, wherein the impurities derive from the drug substance preparation used to prepare the pharmaceutical composition. In another embodiment, the invention provides a pharmaceutical composition having from 0.001%-0.01%, 0.01%-0.1%, or 0.1%-1%, by weight, of the specific product-related impurities, process-related impurities, residual solvents and heavy metals identified herein.

In certain embodiments of this aspect, the invention provides a pharmaceutical composition having a drug substance preparation component containing (S)-(+)-2-(2-fluoro-4-biphenylyl) propionic acid, or a pharmaceutically acceptable salt thereof, as the active ingredient, admixed with one or more pharmaceutically acceptable excipients, wherein the weight of the drug substance preparation is more than about 30%, 35%, 40%, 45%, 50%, or 55% of the total weight of the pharmaceutical composition, and further having limited amounts of the impurities arising from the drug substance preparation described above. In certain embodiments of this embodiment, the drug substance preparation component can be 57% or more, 60% or more, or 63% or more of the total weight of the pharmaceutical composition.

The invention also relates to pharmaceutical compositions and processes for making pharmaceutical compositions that exhibit one or more superior properties relative to other compositions comprising (S)-(+)-2-(2-fluoro-4-biphenylyl) propionic acid, or a pharmaceutically acceptable salt thereof, as the active ingredient. These superior properties include, but are not limited to, one or more of the following: improved bioavailability, improved solubility of the pharmaceutical composition, improved disintegration times for immediate release oral dosage forms, improved dissolution times for immediate release oral dosage forms, decreased tablet friability, increased tablet hardness, improved safety for oral dosage forms, reduced moisture content and/or hygroscopicity for oral dosage forms, improved composition wettability, improved particle size distribution of granules containing the active ingredient, improved composition compressibility, improved composition flow properties, improved chemical stability of the final oral dosage form, improved physical stability of the final oral dosage form, decreased tablet size, improved blend (or composition) uniformity, improved dose uniformity, increased granule density for wet granulated compositions, reduced water requirements for wet granulation, reduced wet granulation time, and/or reduced drying time for wet granulated mixtures.

In still another aspect, the invention provides dosage forms comprising therapeutically effective amounts of (S)-(+)-2-(2-fluoro-4-biphenylyl) propionic acid (or a pharmaceutically acceptable salt thereof) as the active ingredient with limited quantities of impurities arising from the drug substance used to prepare the pharmaceutical compositions used to make these dosage forms. These dosage forms can be designed for oral administration, and, in such instances, may take any acceptable form, including tablets, capsules, caplets, powders, and various granular forms. These dosage forms comprise pharmaceutical compositions that, in turn, comprise the drug substance preparations of the invention, which contain (S)-(+)-2-(2-fluoro-4-biphenylyl) propionic acid, or a salt or ester thereof, as the active ingredient, and limited amounts of impurities, as discussed above. In one embodiment, all of the impurities present in these dosage forms represent about 1%, 0.5%, 0.25% 0.1%, 0.05%, 0.025%, 0.01%, 0.005%, 0.0025%, 0.001%, or less of the total weight of the dosage form as impurities i.e.

[sum of weight(s) of impurities derived from the drug substance, preparation]×100% [total weight of dosage form]

In another embodiment, the invention provides dosage forms having from 1-0.1%, 0.1-0.01%, or 0.01-0.001% of the all the impurities arising from the drug substance preparation, as described above. In another embodiment, the invention provides a dosage form having from 1-0.001%, 0.5-0.001%, 0.25-0.001%, 0.1-0.001%, 0.05-0.001%, 0.025-0.001%, or 0.01-0.001% of one or more impurities arising from the drug substance preparation, as described above.

In certain embodiments of this aspect, the invention provides a unit dosage form having (S)-(+)-2-(2-fluoro-4-biphenylyl) propionic acid, or a salt or ester thereof, as the active ingredient, admixed with one or more pharmaceutically acceptable excipients, wherein the weight of (S)-(+)-2-(2-fluoro-4-biphenylyl) propionic acid, or the salt or ester thereof, is more than about 30%, about 35%, about 40%, about 45%, about 50%, about 55%, about 60%, about 65%, about 70% or about 75% of the total weight of the unit dosage form, and further having a limited amounts of impurities arising from the drug substance preparation used to prepare the pharmaceutical compositions used to prepare the unit dosage form. In certain embodiments, (S)-(+)-2-(2-fluoro-4-biphenylyl) propionic acid, or a salt or ester thereof, can be about 57% or more, about 58% or more, about 59% or more, about 60% or more, about 61% or more, about 62% or more, or about 63% or more of the total weight of the unit dosage form.

The unit dosage form of these embodiments can be provided as a unit dosage form specifically suited for oral administration (e.g. a tablet). This embodiment of the invention is manufactured using a pharmaceutical composition comprising (S)-(+)-2-(2-fluoro-4-biphenylyl) propionic acid, or a salt or ester thereof, as the active ingredient, that has from 30% to 90%, 35% to 90%, 40% to 90%, 45% to 90%, 50% to 90%, or 55% to 90% by weight of the active ingredient, and from 10% to 45% by weight inactive pharmaceutical ingredients, and from 2%-0.001% total (of the total weight of the dosage form) of the impurities arising from the drug substance preparation as described above. In a specific embodiment, the unit dosage form has from 55% to 85% by weight of the active ingredient and 15%-45% by weight inactive pharmaceutical ingredients. In another specific embodiment, the unit dosage form has from 55% to 75% by weight of the active ingredient and from 25% to 45% by weight inactive ingredients. In another specific embodiment, the unit dosage form has from 60% to 70% by weight of the active ingredient and from 30% to 40% by weight inactive pharmaceutical ingredients.

In another embodiment, the invention provides a tablet dosage form having from 5 to 500 mg (S)-(+)-2-(2-fluoro-4-biphenylyl) propionic acid, or therapeutically equivalent amount of a salt or ester thereof

According to this embodiment, the dosage form also has a limited amount of impurities arising from the drug substance preparation, wherein the total weight of these impurities is 1% or less of the total weight of tablet dosage form.

In some embodiment, each tablet has one or more excipients chosen from disintegrants, binders, diluents, glidants, lubricants, colouring agents, stabilizers, preservatives, and/or flavouring agents. In certain embodiments, each tablet has (S)-(+)-2-(2-fluoro-4-biphenylyl) propionic acid, or a salt or ester thereof, and limited amount of impurities arising from the (S)-(+)-flurbiprofen as described above, plus one or more binders, one or more diluents, one or more disintegrants, one or more glidants, one or more lubricants, and if desired, one or more optional ingredients. In certain embodiments, the tablet dosage form is coated.

In a specific embodiment, the invention provides a tablet dosage form containing from about 5 mg to about 500 mg (S)-(+)-2-(2-fluoro-4-biphenylyl) propionic acid, or a molar equivalent amount of a salt or ester thereof, and having about 30%, 35%, 40%, 45%, 50%, or 55%, or more, by weight, of (S)-(+)-2-(2-fluoro-4-biphenylyl) propionic acid in the tablet, and limited amounts of impurities arising from the active ingredient as described above, wherein the total weight of the impurities in the tablet is 0.1% or less of the total weight of the tablet. The tablet dosage forms of this embodiment are specifically suited for oral administration.

In a related embodiment, the unit dosage form is a capsule dosage form. In this embodiment, the capsule dosage form has (S)-(+)-2-(2-fluoro-4-biphenylyl) propionic acid, as the active ingredient, and limited amounts of impurities arising from the active ingredient used to make the pharmaceutical compositions that go into the capsules, and one or more pharmaceutically acceptable excipients as additional components. With a capsule dosage form, the one or more excipients can be chosen from disintegrants, binders, diluents, glidants, lubricants, colouring agents, stabilizers, preservatives, and/or flavouring agents. In certain embodiments, the capsule dosage form comprises a hard gelatin capsule that contains a pharmaceutical composition of the invention.

In a related set of embodiments, the unit dosage form is a caplet dosage form analogous to the tablet or capsule forms as hereinbefore described.

Pharmaceutical compositions may contain, e.g. from about 0.1% to about 99.9%, preferably from about 20% to about 60%, of (S)-(+)-2-(2-fluoro-4-biphenylyl) propionic acid, or a salt or ester thereof, as the active ingredient. Pharmaceutical compositions may be for enteral or parenteral administration are, e.g. those in unit dosage form, such as tablets including sugar-coated tablets, capsules, suppositories and ampoules. These are prepared in a manner known, per se, e.g., by means of conventional mixing, granulating, sugar-coating, dissolving or lyophilizing processes, etc.

The pharmaceutical composition can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers etc.

Typically, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also;
c) binders, e.g., magnesium aluminium silicate, starch paste, gelatin, tragacanth, methylcellulose, sodium carboxymethylcellulose and/or polyvinylpyrrolidone; if desired;
d) disintegrants, e.g., starches, agar, alginic acid or its sodium salt, or effervescent mixtures; and/or
e) absorbents, colorants, flavours and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavouring agents, colouring agents and preserving agents in order to provide pharmaceutically elegant and palatable preparations. Suitable compositions contain (S)-(+)-2-(2-fluoro-4-biphenylyl) propionic acid, or a salt or ester thereof, as the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets may be coated or uncoated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the (5)-(+)-2-(2-fluoro-4-biphenylyl) propionic acid, or a salt or ester thereof, as the active ingredient is mixed with an inert solid diluent, for example, calcium carbonate, calcium phosphate or kaolin, or as soft gelatin capsules wherein the (S)-(+)-2-(2-fluoro-4-biphenylyl) propionic acid, or a salt or ester thereof, as the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure, and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of (5)-(+)-2-(2-fluoro-4-biphenylyl) propionic acid, or a salt or ester thereof, as the active ingredient with carrier. Carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilisers, stabilisers, tonicity enhancing agents, buffers and preservatives.

As used herein a topical application may also pertain to an inhalation or to an intranasal application. They are conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The oral unit dosage forms of the present invention can contain any of the following inactive ingredients, or compounds of a similar nature without limitation: a diluent such as lactose or microcrystalline cellulose; a binder such as hydroxypropyl methylcellulose; a disintegrating agent (disintegrant) such as croscarmellose sodium or microcrystalline cellulose; a lubricant such as magnesium stearate or stearic acid; a glidant such as colloidal silicon dioxide; and optional ingredients such as colouring agents, stabilizers, preservatives and/or flavouring agents or flavour masking agents. In addition, dosage forms of the invention can contain various other materials which modify the physical form of the dosage unit, for example, polymeric coatings (e.g. cellulosics, methacrylates or acrylates), sugar coatings, shellac coatings, colour coatings, wax coatings, or other types of coatings.

In certain embodiments, the invention provides pharmaceutical compositions having (S)-(+)-2-(2-fluoro-4-biphenylyl) propionic acid, or a salt or ester thereof, as the active ingredient, and one or more pharmaceutically acceptable excipients, with (S)-(+)-2-(2-fluoro-4-biphenylyl) propionic acid comprising about 30%, 35%, 40%, 45%, 50%, or 55% or more of the total weight of the unit dosage form. According to these embodiments, the drug substance preparation used in the compositions and dosage forms has less than about 2%, 1%, 0.5%, 0.25% 0.1%, 0.05%, 0.025%, 0.01%, 0.005%, 0.0025%, or 0.001% of the total weight of the pharmaceutical composition as one or more identified product-related impurities, process-related impurities, residual solvents, or heavy metals. In certain specific embodiments of the invention, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2(fluorobiphenyl-4-yl)) propionamide. In other specific embodiments of the invention, the product-related impurities limited to the amounts specified above include those listed above, plus 1-phenylethyl-(2-(2-fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals.

The unit dosage form of these embodiments of the invention is suited for gastrointestinal administration by an oral route (e.g. a tablet to be taken by mouth; oral administration). In some of these embodiments, (S)-(+)-2-(2-fluoro-4-biphenylyl) propionic acid is present as 57% or more, 60% or more, or 63% or more of the total weight of the of the unit dosage form. In some of these embodiments, the unit dosage form has about 5 mg, 10 mg, 25 mg, 50 mg, 100 mg, 250 mg, 500 mg, or more, of (S)-(+)-2-(2-fluoro-4-biphenylyl) propionic acid in the free acid form (or a therapeutically equivalent amount of a salt or ester thereof) contained within each unit dosage form (i.e., tablet). In one specific embodiment, approximately 400 mg of (S)-(+)-2-(2-fluoro-4-biphenylyl) propionic acid is present in a tablet dosage form as the free acid, and comprises from 65% to 68% of the total weight of the tablet dosage form.

In other embodiments of this aspect of the invention, the invention provides an (S)-(+)-2-(2-fluoro-4-biphenylyl) propionic acid-containing tablet dosage forms having from 55% to 90% by weight (S)-(+)-2-(2-fluoro-4-biphenylyl) propionic acid and from 10% to 45% by weight inactive pharmaceutical ingredients. According to this embodiment, the drug substance preparation used in preparing the tablet dosage forms has less than about 2%, 1%, 0.5%, 0.25% 0.1%, 0.05%, 0.025%, 0.01%, 0.005%, 0.0025%, or 0.001% of the total weight of the drug substance as one or more identified product-related impurities, process-related impurities, residual solvents, or heavy metals. In certain specific embodiments of the invention, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2(fluorobiphenyl-4-yl)) propionamide. In other specific embodiments of the invention, the product-related impurities limited to the amounts specified above include those listed above, plus 1-phenylethyl-(2-2(fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals. In these embodiments, the tablet dosage form is specifically designed for oral administration.

In other embodiments of this aspect of the invention, the (S)-(+)-2-(2-fluoro-4-biphenylyl) propionic acid-containing tablet dosage form has from 55% to 85% by weight (S)-(+)-2-(2-fluoro-4-biphenylyl) propionic acid, and from 15% to 45% by weight inactive pharmaceutical ingredients. In still other embodiments of this aspect of the invention, the (S)-(+)-2-(2-fluoro-4-biphenylyl) propionic acid-containing tablet dosage form has from 55% to 75% by weight (S)-(+)-2-(2-fluoro-4-biphenylyl) propionic acid, and from 25% to 45% inactive ingredients. In still other embodiments of this aspect of the invention, the (S)-(+)-2-(2-fluoro-4-biphenylyl) propionic acid-containing tablet dosage form has from 60% to 70% by weight (S)-(+)-2-(2-fluoro-4-biphenylyl) propionic acid and from 30% to 40% inactive pharmaceutical ingredients. According to one specific embodiment of this aspect of the invention, the tablet dosage form has from 55% to 90% by weight (S)-(+)-2-(2-fluoro-4-biphenylyl) propionic acid, 1% to 20% by weight lactose (calculated based on anhydrous lactose), 1% to 20% by weight hydroxypropyl methylcellulose, 5% to 45% by weight microcrystalline cellulose, and, if desired, optional ingredients.

The (S)-(+)-2-(2-fluoro-4-biphenylyl) propionic acid-containing unit dosage forms of the present invention generally have 55% or more of the total weight of the unit dosage form as (S)-(+)-2-(2-fluoro-4-biphenylyl) propionic acid, with the remaining weight comprised of one or more pharmaceutically acceptable excipients. According to these embodiment, the drug substance used in the compositions used to manufacture the unit dosage forms has less than about 2%, 1%, 0.5%, 0.25% 0.1%, 0.05%, 0.025%, 0.01%, 0.005%, 0.0025%, or 0.001% of the total weight of the drug substance as one or more identified product-related impurities, process-related impurities, residual solvents, or heavy metals. In specific embodiments, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2(fluorobiphenyl-4-yl)) propionamide. In other specific embodiments of the invention, the product-related impurities limited to the amounts specified above include those listed above, plus 1-phenylethyl-(2-2(fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals.

The excipients used to prepare the unit dosage forms of the invention include one or more excipients chosen from disintegrants, binders, diluents, glidants, and lubricants, as well as any desired optional ingredient. Thus, in one set of embodiments of the invention, the unit dosage form has an excipient that is a disintegrant (e.g. microcrystalline cellulose and/or croscarmellose). The amount of disintegrants in the dosage form of the invention can be 45% or less, 40% or less, 35% or less, 30% or less, or less than 25% of the total weight of the unit dosage form. In another set of embodiments of the invention, the unit dosage form has an excipient that is a binder (e.g. hydroxypropyl methylcellulose). The amount of binder in the dosage form can be 20% or less, 15% or less, 10% or less, or less than 8% of the total weight of the unit dosage form. In yet another set of embodiments of the invention, the unit dosage form has an excipient that is a diluent such as lactose. The amount of diluent in the unit dosage form can be 20% or less, 17% or less, 15% or less, or less than 12% of the total weight of the unit dosage form. In still another set of embodiments of the invention, the unit dosage form has an excipient that is a glidant such as colloidal silicon dioxide. The amount of glidant in the unit dosage form can be 7% or less, 5% or less, 3% or less, or less than 2% of the total weight of the unit dosage form. In another set of embodiments of the invention, the unit dosage form has an excipient that is a lubricant such as magnesium stearate. The amount of lubricant in the unit dosage form can be 10% or less, 5% or less, 3% or less, or less than 2% of the total weight of the unit dosage form.

In another set of embodiments of the invention, the unit dosage form, containing (S)-(+)-2-(2-fluoro-4-biphenylyl) propionic acid, or a salt or ester thereof, and one or more excipients, is coated. In one set of embodiments of the invention, the weight of the coating (e.g. Opadry Pink) is from 0.1% to 10% of the total weight of the unit dosage form. In another set of embodiments, the weight of the coating is from 0.1% to 8% of the total weight of the unit dosage form. In another set of embodiments, the weight of the coating is from 0.1% to 5% of the total weight of the unit dosage form.

In certain embodiments, the invention also provides a dosage form having from about 5 mg to 500 mg (S)-(+)-2-(2-fluoro-4-biphenylyl) propionic acid, or a therapeutically equivalent amount of a salt or ester thereof. According to these embodiments, the drug substance preparation used in the dosage forms has less than about 2%, 1%, 0.5%, 0.25% 0.1%, 0.05%, 0.025%, 0.01%, 0.005%, 0.0025%, or 0.001% of the total weight of the drug substance as one or more identified product-related impurities, process-related impurities, residual solvents, or heavy metals. In specific embodiments, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2(fluorobiphenyl-4-yl)) propionamide. In other specific embodiments of the invention, the product-related impurities limited to the amounts specified above include those listed above, plus 1-phenylethyl-(2-2(fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals. The dosage forms of this embodiment can be a unit dosage form suited for oral administration (e.g., a tablet).

The present invention also relates to (S)-(+)-2-(2-fluoro-4-biphenylyl) propionic acid-containing unit dosage forms having 55% or more by weight of (S)-(+)-2-(2-fluoro-4-biphenylyl) propionic acid, or a salt or ester thereof. According to an embodiment of this aspect of the invention, the drug substance used in the pharmaceutical compositions and dosage forms has less than about 2%, 1%, 0.5%, 0.25% 0.1%, 0.05%, 0.025%, 0.01%, 0.005%, 0.0025%, or 0.001% of the total weight of the drug substance as one or more identified product-related impurities, process-related impurities, residual solvents, or heavy metals. In specific embodiments, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2-(fluorobiphenyl-4-yl)) propionamide. In other specific embodiments of the invention, the product-related impurities limited to the amounts specified above include those listed above, plus 1-phenylethyl-(2-2(fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals.

As used herein, substantially bioequivalent refers to Cₘₐₓ (maximum plasma concentration) and AUC (area under the curve; drug exposure) parameters within 80% to 125% of the reference parameter. The unit dosage forms of these embodiments are suited for oral administration (e.g. a tablet), and in certain embodiments, the unit dosage form is a coated tablet.

In one embodiment, oral administration, to a fasting subject, of a single dose (e.g. two tablets each having 250 mg (S)-(+)-flurbiprofen as the active ingredient of the dosage forms of the present invention, provides a Cₘₐₓ of about 25-200 μg per mL per dose; preferably 25-150 μg per mL per dose; and more preferably, between 30-95 μg per mL per dose. According to the embodiments of this aspect of the invention, the drug substance used in the compositions and dosage forms also has less than about 2%, 1%, 0.5%, 0.25% 0.1%, 0.05%, 0.025%, 0.01%, 0.005%, 0.0025%, or 0.001% of the total weight of the drug substance as one or more identified product-related impurities, process-related impurities, residual solvents, or heavy metals. In specific embodiments, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2(fluorobiphenyl-4-yl)) propionamide. In other specific embodiments of the invention, the product-related impurities limited to the amounts specified above include those listed above, plus 1-phenylethyl-(2-2(fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals.

In some embodiments of this aspect of the invention, oral administration of a single dose of a dosage form of the invention to a fasting subject, provides a Cₘₐₓ, per dose, of greater than about 25 μg per mL, 30 μg per mL, 35 μg per mL, 40 μg per mL, 45 μg per mL, 50 μg per mL, 55 μg per mL, or 60 μg per mL. Administration of a single dose of a dosage form of the invention to a fasting subject provides an AUC (area under curve of concentration versus time; total drug exposure) of from about 200 hr μg/mL to about 600 hr μg/mL.

Desirably, the dosage forms of the present invention are substantially free of (R)-(−)-2-(2-fluoro-4-biphenylyl) propionic acid. Therefore, in embodiments of dosage forms of the present invention, (S)-(+)-2-(2-fluoro-4-biphenylyl) propionic acid is present in significant excess over (R)-(−)-2-(2-fluoro-4-biphenylyl) propionic acid. In certain embodiments, (S)-(+)-2-(2-fluoro-4-biphenylyl) propionic acid is present in an enantiomeric excess of about 98.0%, 98.5%, 99.0%, 99.1%, 99.2%, 99.3%, 99.4%, 99.5%, 99.6%, 99.7%, 99.8%, or 99.9%, or more. Therefore, in these embodiments of dosage forms of the present invention, (R)-(−)-2-(2-fluoro-4-biphenylyl) propionic acid is present in less than about 1%, 0.75%, 0.5%, 0.45%, 0.4%, 0.35%, 0.3% 0.25%, 0.2%, 0.15%, 0.1%, or 0.05%, or less, by weight, of the total amount of (S)-(+)-2-(2-fluoro-4-biphenylyl) propionic acid and (R)-(−)-2-(2-fluoro-4-biphenylyl) propionic acid present in the dosage form.

In one set of preferred embodiments of the invention, a tablet unit dosage form is provided having from about 5 mg to 500 mg (S)-(+)-2-(2-fluoro-4-biphenylyl) propionic acid (or a bioequivalent amount of a salt or ester thereof), from about 50 mg to 70 mg lactose, from about 3 mg to 7 mg colloidal silicon dioxide, from about 30 mg to 50 mg hydroxypropyl methylcellulose, from about 70 mg to 105 mg microcrystalline cellulose, from about 1 mg to 5 mg croscarmellose sodium, from about 4 mg to 8 mg magnesium stearate, and optional ingredients as desired. In this preferred set of embodiments, the drug substance used to prepare the tablet unit dosage form has less than about 2%, 1%, 0.5%, 0.25% 0.1%, 0.05%, 0.025%, 0.01%, 0.005%, 0.0025%, or 0.001% of the total weight of the drug substance as one or more identified product-related impurities, process-related impurities, residual solvents, or heavy metals. In specific embodiments, the product-related impurities limited to the amounts specified above include (R)-(−)-2-(2-fluoro-4-biphenylyl) propionic acid; 2-(4-biphenylyl) propionic acid; and methyl(2-(2-fluoro-4-biphenylyl)) propionate. In other specific embodiments of the invention, the product-related impurities limited to the amounts specified above include those listed above, plus 1-phenylethyl-(2-2(fluorobiphenyl-4-yl)) propionamide. In other specific embodiments of the invention, the product-related impurities limited to the amounts specified above include those listed above, plus 1-phenylethyl-(2-2(fluorobiphenyl-4-yl)) propionamide and 2-(2-fluorobiphenyl-4-yl) propionamide. In other specific embodiments of the invention, the process-related impurities limited to the amounts specified above include (S)-(−)-α-methylbenzylamine. In other specific embodiments of the invention, the process-related impurities limited to the amounts specified above include (S)-(−)-α-methylbenzylamine and the residual solvents, including toluene, methanol and n-heptane. In yet other specific embodiments of the invention, the process-related impurities limited to the amounts specified above include (S)-(−)-α-methylbenzylamine; the residual solvents listed above, and heavy metals.

Base addition salts may be produced by reacting with free bases in known manner. A pharmaceutically acceptable salt is any salt of (S)-(+)-2-(2-fluoro-4-biphenylyl) propionic acid that is suitable for administration to an animal or human. A pharmaceutically acceptable salt also refers to any salt which may form in vivo as a result of administration of an acid, another salt, or a prodrug which is converted into SFP or salt. A salt comprises one or more ionic forms of the compound, such as a conjugate acid or base, associated with one or more corresponding counter-ions.

As used herein, the term “salts” shall mean “pharmaceutically acceptable salts” and refers to salts that retain the biological effectiveness and properties of the compounds of this invention and, which are not biologically or otherwise undesirable. Similarly, the “esters” shall mean “pharmaceutically acceptable esters”.

Salts are desirably pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminium, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. The pharmaceutically acceptable salts of the present invention can be synthesized from a parent (S)-(+)-2-(2-fluoro-4-biphenylyl) propionic acid, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the (S)-(+)-2-(2-fluoro-4-biphenylyl) propionic acid. Such reactions are typically carried out in water or in an organic solvent, or in a mixture of the two. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Other salts include ammonium or amino acid salts which are water soluble thereby being preferred. Complex salts with basic amino acids can be used directly and mixed salts with neutral or acidic amino acids are previously converted into the alkali metal, alkaline earth metal or ammonium salts. A preferred amino acid is lysine. Other methods also known for medicaments, in which the active material is adsorbed onto aluminium oxide gels, can also be carried out with the flurbiprofen according to the present invention. The (S)-(+)-flurbiprofen salts produced can then be further worked up in the manner described hereinbefore. Preferably, the flurbiprofen salts are prepared indirectly by adding the bases required for the salt formation. Amino acid salts may comprise an essential amino acid, such as, histidine, isoleucine, leucine, lysine, methionine, phenylalanine, threonine, tryptophan, valine and tyrosine; or a non-essential amino acid, such as, alanine, arginine, aspartate, cysteine, glutamate, glutamine, glycine, proline, serine, asparagines and selenocysteine. Alternatively, the salt may comprise an amino sugar, such as meglumine.

Esters will generally be pharmaceutically acceptable esters which may be produced from the FPB reacted with an appropriate alcohol. Esters of pharmaceutically acceptable acids can be formed with organic acids, e.g. acetate, acetoxyethyl ester (axetil), aspartate, benzoate, besylate, camsylate, cinnamate, citrate, edisylate, esylate, ethanesulfonate, formate, fumarate, gluceptate, gluconate, glucuronate, glycolate, hexafluorophosphate, hibenzate, isethionate, lactate, malate, maleate, malonate, mandelate, mesylate, methanesulfonate, methylsulphate, 2-napsylate, naphthylate, nicotinate, orotate, oxalate, palmitate, pamoate, propionate, pyruvate, saccharate, salicylate, stearate, succinate, tartrate, p-toluenesulfonate, tosylate, trifluoroacetate, and the like.

Although the foregoing invention has been described in some detail by way of illustration and example for purposes of clarity of understanding, it will be obvious that certain changes and modifications may be practiced within the scope of the appended claims.

### Method for Preparing (S)-(+)-2-(2-Fluoro-4-Biphenylyl) Propionic Acid

(S)-(+)-flurbiprofen was selectively isolated from racemic sodium flurbiprofen dihydrate, by modifying the method disclosed in International application No. WO 2008/095186, which is incorporated herein by reference. The quality of material obtained via this method was then examined.

Sodium flurbiprofen dihydrate (200.0 g) was dissolved in toluene (370 mL) and hydrochloric acid (37%, 80 mL in 133 mL H₂O), and the mixture was heated to 60° C. for 30 mins. The aqueous layer was then removed, and the organic layer was washed with hot water (108 mL). Methanol (94 mL) was then added and mixture heated to 60° C. (S)-(+)-α-methylbenzylamine (35.2 g) in toluene (71.5 mL) was added dropwise over 30 mins. The solution was stirred at 60° C. for 30 mins, then warmed to 68° C. and held for 30 mins. The solution was cooled to 50° C., then further cooled to 3° C. and held for 1 hour. Crystals of (S)-(+)-flurbiprofen-(S)-(−)-α-methylbenzylamine were isolated, washed with cold toluene (53 mL) and dried on a vacuum filter overnight.

The crystals of (S)-(+)-flurbiprofen-(S)-(−)-α-methylbenzylamine (49.4 g) were returned to the vessel and suspended in methanol (125 mL) and toluene (500 mL). The mixture was warmed to 70° C., stirred for 15 mins, then cooled to 55° C. and stirred for 30 mins. The solution was then warmed to 60° C. for 30 mins, slowly cooled to 50° C., then further cooled to 3° C. and stirred for 1 hour. Crystals of (S)-(+)-flurbiprofen-(S)-(−)-α-methylbenzylamine were isolated, washed with cold toluene (53 mL) and dried on a vacuum filter overnight.

The crystals of (S)-(+)-flurbiprofen-(S)-(−)-α-methylbenzylamine (38.4 g) were dissolved in deionised water (38 mL), toluene (160 mL) and aqueous hydrochloric acid (37%, 16 mL), and stirred at 60° C. for 30 mins. The resulting solution was transferred hot to a separating funnel and the aqueous layer was removed. The organic layer was washed with water (50 mL) and aqueous hydrochloric acid (37%, 2 mL), then further water (2×50 mL). The organic layer was then heated to partially remove the solvent and reduce the volume to approx. 100 mL. The resulting solution was then cooled to 30° C., and stirred for 30 mins. The solution was slowly cooled to 0° C., and the resulting crystals stirred for 1 hr. The crystalline (S)-(+)-flurbiprofen was then filtered, washed with cold n-heptane (40 mL) and dried overnight under vacuum at 60° C. Yield 19.9 g (12.5%).

TABLE 1
Analysis of Final Product

| | |
|---|---|
| Test | Amount |

Impurities by HPLC:

| | |
|---|---|
| 2-(4-biphenylyl)propionate | 0.09% |
| methyl (2-(2-fluoro-4-biphenylyl)) | None detected |
| propionate | |
| 1-phenylethyl-(2-(2-fluorobiphenyl- | None detected |
| 4-yl))propionamide | |
| Other identified impurities | None detected |
| Other unidentified impurities | None detected |
| Total | 0.09% |
| α-methylbenzylamine | 16 ppm |
| Enantiomeric purity | 99.2% |

1. (canceled)

2. A pharmaceutical composition comprising (S)-(+)-2-(2-fluoro-4-biphenylyl) propionic acid, or a salt or ester thereof, as the active ingredient, and having substantially limited amounts of specific impurities associated with the synthesis and purification of the active ingredient and from about 0.001% and about 3%, by weight, product-related impurities, wherein said product-related impurities comprises not more than about 0.5%, by weight 2-(4-biphenylyl) propionic acid, or a salt thereof.

3. (canceled)

4. A pharmaceutical composition according to claim 2 wherein said product-related impurities comprises not more than about 2%, by weight, of the enantiomeric impurity (R)-(−)-2-(2-fluoro-4-biphenylyl) propionic acid, or a salt thereof.

5. A pharmaceutical composition according to claim 2 wherein said product-related impurities further comprise not more than about 0.1%, by weight, methyl(2-(2-fluoro-4-biphenylyl)) propionate, or a salt thereof.

6. A pharmaceutical composition according to claim 2 wherein said product-related impurities comprise not more than about 1%, by weight, of the enantiomeric impurity (R)-(−)-2-(2-fluoro-4-biphenylyl) propionic acid, or a salt thereof.

7. A pharmaceutical composition according to claim 2 wherein said product-related impurities comprise not more than about 0.5%, by weight, of the enantiomeric impurity (R)-(−)-2-(2-fluoro-4-biphenylyl) propionic acid, or a salt thereof.

8. A pharmaceutical composition according to claim 2 comprising (R)-(−)-2-(2-fluoro-4-biphenylyl) propionic acid, or a salt or ester thereof, and one or more excipients.

9. A pharmaceutical composition according to claim 2 one or more additional excipients.

10. A pharmaceutical composition according to claim 2 wherein the composition is a tablet formulated to orally administer from about 50 mg to about 500 mg of (S)-(+)-2-(2-fluoro-4-biphenylyl) propionic acid, or the molar equivalent amount of a salt or ester thereof.

11. A pharmaceutical composition according to claim 2 wherein the composition comprises an adhesive preparation obtained by coating one surface of support with an adhesive which contains (S)-(+)-2-(2-fluoro-4-biphenylyl) propionic acid, or a salt or ester thereof, and a plaster base.

12. A pharmaceutical composition according to claim 11 comprising a patch for topical or transdermal administration, such that the (S)-(+)-2-(2-fluoro-4-biphenylyl) propionic acid, or a salt or ester thereof, is present on an adhesive support in a density of from about 50 to about 1000 μm/cm².

13-15. (canceled)

16. A pharmaceutical composition according to claim 2 further comprising not more than about 1,000 ppm of the process-related impurity, (S)-(−)-α-methylbenzylamine.

17-26. (canceled)

27. A drug substance preparation comprising:
(1) (S)-(+)-2-(2-fluoro-4-biphenylyl) propionic acid, or a pharmaceutically acceptable salt or ester thereof, as the active pharmaceutical ingredient; and
(2) between about 0.001% and about 2%, by weight, product-related impurities.

28. A drug substance preparation according to claim 27 wherein said product-related impurities comprises not more than about 0.5%, by weight, 2-(4-biphenylyl) propionic acid.

29. (canceled)

30. A drug substance preparation according to claim 27 wherein said product-related impurities comprises not more than about 1.0%, by weight, of the enantiomeric impurity (R)-(−)-2-(2-fluoro-4-biphenylyl) propionic acid, or a salt thereof.

31. A drug substance preparation according to claim 27 wherein said product-related impurities comprises not more than about 1,000 ppm of the process-related impurity, (S)-(−)-α-methylbenzylamine.

32-35. (canceled)

36. A drug substance preparation according to claim 27 wherein said product-related impurities comprises not more than about 0.5%, by weight, of the enantiomeric impurity (R)-(−)-2-(2-fluoro-4-biphenylyl) propionic acid, or a salt thereof.

37. (canceled)

38. A method of treatment or alleviation of inflammation and/or pain which comprises administering a therapeutically effective amount of (S)-(+)-2-(2-fluoro-4-biphenylyl) propionic acid, or a salt or ester thereof, having substantially limited amounts of specific impurities associated with the synthesis and purification of the (S)-(+)-2-(2-fluoro-4-biphenylyl) propionic acid, to a patient in need of such treatment.

39-40. (canceled)

41. A process for the preparation of (S)-(+)-2-(2-fluoro-4-biphenylyl) propionic acid, or a salt or ester thereof, and having substantially limited amounts of specific impurities associated with the synthesis and purification of the (S)-(+)-2-(2-fluoro-4-biphenylyl) propionic acid, said process comprising:
(i) reacting a solution of flurbiprofen and (S)-(−)-α-alkyl(C₁₋₆)benzylamine;
(ii) isolating (S)-(+)-flurbiprofen-(S)-(−)-α-alkyl(C₁₋₆)benzylamine formed in step (i);
(iii) optionally recrystallising the (S)-(+)-flurbiprofen-(S)-(−)-α-alkyl(C₁₋₆)benzylamine;
(iv) reacting an organic solution of (S)-(+)-flurbiprofen-(S)-(−)-α-alkyl(C₁₋₆)benzylamine with an acid to form (S)-(+)-flurbiprofen;
(v) separating the organic portion containing (S)-(+)-flurbiprofen;
(vi) optionally washing the organic portion with a further acid; and
(vii) isolating the crystalline (S)-(+)-flurbiprofen.

42. (canceled)
